(19) United States
(12) Patent Application Publication
(10) Pub. No.: US 2021/0030021 A1
(43) Pub. Date: Feb. 4, 2021

(54) METHODS AND SYSTEMS FOR PROCESSING A MICROCROP TO GENERATE NUTRITIONALLY DENSE CONSUMER PRODUCTS

(71) Applicant: PARABEL NUTRITION, INC., Vero Beach, FL (US)

(72) Inventors: Peter Sherlock, Indialantic, FL (US); Ebenezer Ifeduba, Palm Bay, FL (US)

(21) Appl. No.: 16/882,106

(22) Filed: May 22, 2020

Related U.S. Application Data
(60) Provisional application No. 62/882,440, filed on Aug. 2, 2019.

(51) Int. Cl. A23J 1/00; A01G 22/00; A01J 11/00; A23J 3/14

(52) U.S. Cl. CPC A23J 1/006; A23J 3/14; A01J 11/00; A01G 22/00

(57) ABSTRACT

The present disclosure relates, according to some embodiments, to method of generating multiple products from a microcrop. A first juice, derived from a microcrop and containing microcrop protein, may be processed to generate products including a microcrop milk, a milk base, a milk mixture, a dried base powder, a green food product, and a colorless protein product. A first juice may be hydrolyzed and filtered to generate each of a decolored permeate and a green retentate which are, in turn, further processed into products. Use of a decolored permeate to generate products allows for consumer food products which contain microcrop protein but lack green coloration. Compositions of the products may be optimized through a combination of demineralization, drying, dewatering, mixing, formulating, and anti-microbial treatments including pasteurization. Polyphenol concentration of products may be reduced through adsorption and clarification.

### CROSS REFERENCE TO RELATED APPLICATIONS

This application claims priority under 35 U.S.C. § 119(e) to U.S. Provisional Application No. 62/882,440, filed on Aug. 2, 2019, which is hereby incorporated by reference in its entirety.

### FIELD OF THE DISCLOSURE

The present disclosure relates, in some embodiments to methods and systems for generating multiple products from a microcrop (e.g., aquatic plant species, Lemna) including a green food product, a green fiber product, a colorless protein product, an electrolyte drink, a dried base powder, and a microcrop milk.

### BACKGROUND OF THE DISCLOSURE

An ever-increasing global population continues to fuel a plethora of sustainability concerns including sufficient and affordable access to protein sources for human consumption, including mammalian meat and milk products. Dairy cattle in particular are a known contributor to increased global carbon emissions. Many consumers also desire alternative protein products that are not derived from mammals due to health, allergy, and/or ethical concerns.

Besides sustainability issues, nutritionally viable mammalian alternatives are needed that contain adequate protein having a desirable amino acid profile and/or high fiber content. Many plant-based protein products have inferior amino acid profiles. In contrast, a microcrop protein source can be used to generate mammalian protein product alternatives having desirable amounts of protein with an enhanced amino acid profile containing all essential amino acids. Another issue with obtaining plant-based protein is that plants have low nominal protein amounts, making it an inefficient endeavor due to the low protein yield. These low yields from plants make the protein isolation process an arduous task while concurrently producing large quantities of waste products.

In addition, vitamin B12 deficiency is prevalent in modern society and can result is serious health concerns including anemia. Traditional sources of vitamin B12 include fish, meat, poultry, eggs, mammalian milk, and mammalian milk products. However, each of these sources of vitamin B12 is riddled with the same sustainability concerns discussed above. Further, individuals with a vegan diet typically resort to supplementing their diets with a synthetic form of Vitamin B12 (e.g., Cyanocobalmin) which, unlike its naturally occurring counterparts (e.g., Adenosylcobalmin, Hydroxycobalmin, and Methylcobalmin), is not a bioactive form.

### SUMMARY

The present disclosure relates, according to some embodiments, to generating multiple products from a microcrop. These products may comprise a microcrop protein and may include, without limitation, a first juice, a microcrop milk, a dried base powder, a green food product, a colorless protein product, a milk mixture, a milk base, a decolored permeate, and a green retentate.

A first juice may be generated by: lysing a microcrop to generate a lysed biomass; separating a lysed biomass to generate a solid fraction and a juice fraction; and separating a juice fraction to generate a first juice. In some embodiments, a lysed biomass may be hydrolyzed prior to separating it into a solid fraction and a juice fraction.

Each of a decolored permeate and a green retentate, according to some embodiments, may be generated from a first juice by: hydrolyzing a first juice to generate a hydrolyzed protein juice; and filtering a hydrolyzed protein juice to generate at least one of a decolored permeate and a green retentate. In some embodiments, a first juice may be treated and cooled to an incubation temperature prior to hydrolysis. A treatment may be one or more processes, including pasteurization, heating to specific temperatures and maintaining said temperature for a specific time, and gamma ray exposure. In other embodiments, a first juice may be heated to an incubation temperature prior to hydrolysis.

A milk mixture, according to some embodiments, may be generated from a decolored permeate by: demineralizing the decolored permeate to generate a demineralized stream; dewatering the demineralized stream to generate a dewatered demineralized stream; and using at least a portion of the dewatered demineralized stream as a milk mixture. In some embodiments, a milk mixture may include at least a portion of the dewatered demineralized stream that is mixed with at least a portion of the demineralized stream. Polyphenols may be reduced in a decolored permeate prior to demineralization using one or more of an adsorption process and a clarification process.

A milk base may be generated by dewatering a milk mixture. A microcrop milk may be generated by formulating the milk base. Formulating, according to some embodiments, may be amending a milk base with a fat component and an emulsifier. Any one or more of a milk mixture and a milk base may be dried to generate a dried base powder.

A green food product may be generated from a green retentate by: dewatering the green retentate to generate a dewatered green retentate; and drying the dewatered green retentate to generate a green food product. In some embodiments, one or more of a green retentate and a dewatered green retentate may be treated prior to drying by at least one of pasteurization, heating to specific temperatures and maintaining said temperature for a specific time, and gamma ray exposure.

A colorless protein product may be generated from a decolored permeate by: demineralizing the decolored permeate to generate a demineralized stream; dewatering the demineralized stream to generate a dewatered demineralized stream; and drying the dewatered demineralized stream to generate a colorless protein product. Polyphenols may be reduced in a decolored permeate prior to demineralization using one or more of an adsorption process and a clarification process. A dewatered demineralized stream may be treated prior to drying by at least one of pasteurization, heating to specific temperatures and maintaining said temperature for a specific time, and gamma ray exposure.

### BRIEF DESCRIPTION OF THE DRAWINGS

Some embodiments of the disclosure may be understood by referring, in part, to the present disclosure and the accompanying drawings, wherein:

FIG. 1A is a flow diagram illustrating a system for processing a microcrop to generate a first juice and a green fiber product, according to a specific example embodiment of the disclosure; and

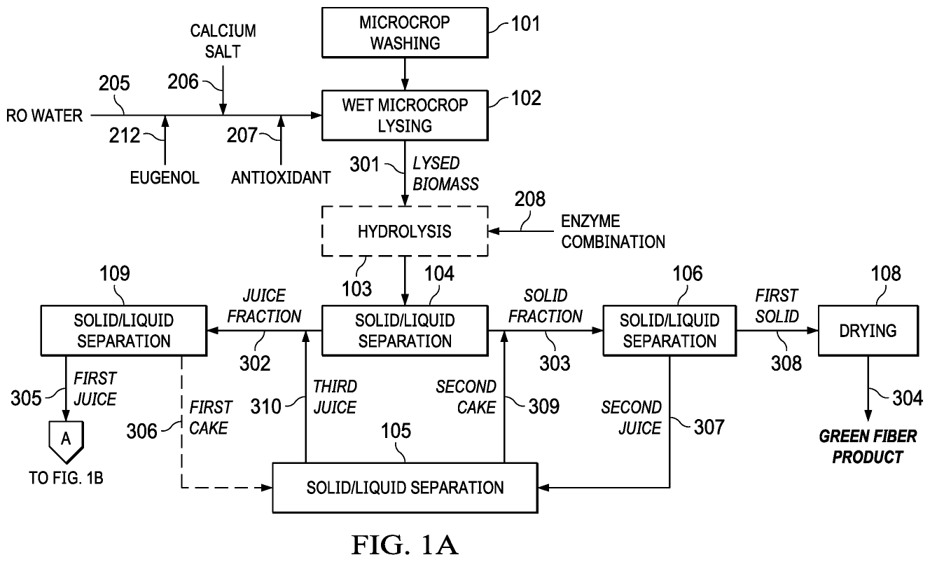

FIG. 1B is a flow diagram that further outlines a system for processing a microcrop beginning with a first juice of FIG. 1A, according to a specific example embodiment of the disclosure.

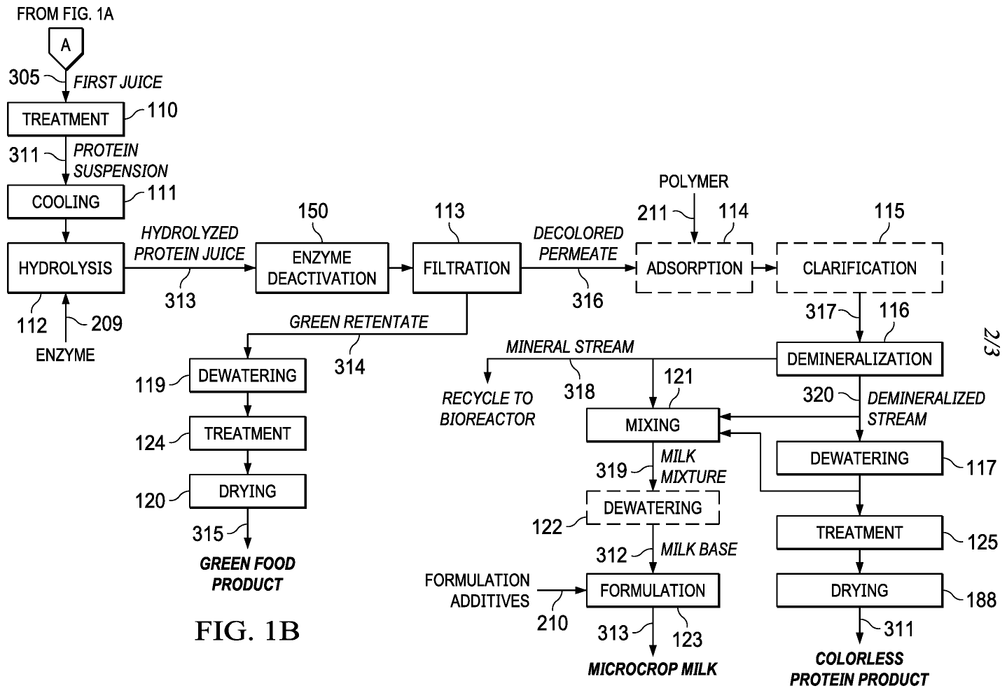

FIG. 1C is a flow diagram that further outlines a system for processing a microcrop beginning with a milk mixture and a milk base of FIG. 1B, according to a specific example embodiment of the disclosure.

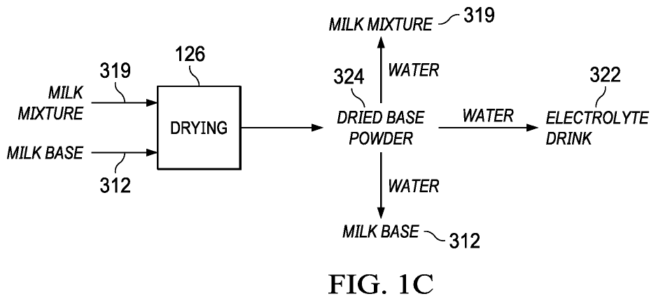

### DETAILED DESCRIPTION

The present disclosure relates, in some embodiments, to methods and systems of processing a microcrop to yield multiple products, the multiple products including a green food product, a green fiber product, a colorless protein product, an electrolyte drink (e.g., protein-rich), and a microcrop milk. The present disclosure further relates to the multiple products derived from the described methods and systems.

A microcrop may comprise a single floating aquatic species (e.g., Lemna species, Salvinia species). A microcrop may include species of Lemna (e.g., duckweed, water lentil), Spirodela, Landoltia, Wolfiella, Salvinia (e.g., floating fern), Wolffia (e.g., watermeal), Azolla (e.g., mosquito fern), Pistia (e.g., water lettuce), or any combination thereof. According to some embodiments, a microcrop may be a species of Lemna, for example, Lemna minor, Lemna obscura, Lemna minuta, Lemna gibba, Lemna valdiviana, or Lemna aequinoctialis. A microcrop may comprise, according to some embodiments, a combination of two or more floating aquatic species (e.g., two species of Lemna, a species of Lemna and a species of Wolfiella). In some embodiments, a microcrop may be selected from a local floating aquatic species based on identified compositional and growth characteristics that have developed within the local environmental conditions. Local species may out-compete other species in open ponds or bioreactors based on their adaptation to the local environmental conditions. A microcrop, in some embodiments, may be adjusted in response to seasonal variations in temperature and light availability.

A microcrop may have characteristics that are advantageous in comparison to other aquatic species (e.g., rapid growth rate; reduced nutritional requirements; ease of harvesting and/or processing; enhanced amino acid profile; enhanced palatability; reduced evapotranspiration rate; increased protein composition, reduced oxalic acid content). For example, Lemna is a genus of free-floating aquatic plants from the Lemnaceae family (e.g., duckweed) that grow rapidly. Lemna protein has an essential amino acid profile that more closely resembles animal protein than most other plant proteins. Table 1 shows a typical essential amino acid compositional profile of Lemna protein. Additionally, Lemna provides high protein yields, with freshly harvested Lemna containing up to about 43% protein by dry weight. Furthermore, Lemna leaves have about 5% total solids (with about 40% of the solids being carbohydrates consisting predominantly of soluble and insoluble fiber) and are highly digestible, even for monogastric animals.

TABLE 1
Essential Amino Acid Profile of Lemna Protein
Concentration

| Essential Amino Acid | Protein (g/100 g) |
|---|---|
| Lysine | 5.9 |
| Leucine | 9.7 |
| Isoleucine | 5.1 |
| Methionine | 2.4 |
| Phenylalanine | 6.3 |
| Threonine | 4.4 |
| Tryptophan | 2.0 |
| Valine | 6.3 |
| Histidine | 2.7 |
| Arginine* | 6.8 |

*Conditionally non-essential amino-acid.

Microcrop Cultivation, Harvesting, and Preliminary Processing

The present disclosure further relates to the cultivation, harvesting, and preliminary processing of a microcrop that may be further processed to generate at least one of a green food product, a green fiber product, a colorless protein product, an electrolyte drink, and a microcrop milk. A detailed description of processes by which a microcrop may be cultivated, harvested, preliminarily processed, lysed, and separated, in accordance with some embodiments of the present disclosure, can be found in U.S. patent application Ser. No. 16/803,792, U.S. Pat. No. 8,679,352, U.S. patent application Ser. No. 15/179,963, U.S. patent application Ser. No. 15/751,826, and U.S. patent application Ser. No. 15/263,253, each of which is incorporated herein by reference in its entirety as set forth in full. However, persons skilled in the art would appreciate that any number of methods may be used to generate a first juice and this disclosure is not limited to those explicitly described herein or incorporated by reference. As one example of the present disclosure, a detailed description of the processes by which a microcrop of the present disclosure may be harvested is found in U.S. patent application Ser. No. 16/803,792 (i.e., the '792 Application), which is incorporated herein by reference in its entirety as set forth in full. As disclosed in the '792 application, a method of continuously supplying a harvested biomass comprising a floating aquatic plant species to a processing facility may comprise cultivating a microcrop (e.g., a floating aquatic plant species) in a bioreactor system, harvesting the microcrop to generate a harvested biomass, and conveying the harvested biomass to a first position of a harvest canal to form a conveyed biomass. A harvest canal may include a trough configured to contain the conveyed biomass in a volume of a medium, a canopy configured to provide at least an 80% reduction in solar radiation compared to the external surface of the canopy, and a propulsion mechanism configured to impart a motion on the first medium such that the harvested biomass may be transported from the first position to the second position within the harvest canal. The harvest canal may be positioned adjacent to an outer perimeter of bioreactor system and form an infinity loop. The method may further include activating the propulsion mechanism to impart motion on the first medium and propel the harvested biomass from the first position to the second position, and transferring at least a portion of the propelled biomass from the second position of the harvest canal to a processing facility. The propelled biomass of the '792 application may comprise a microcrop of the instant disclosure, in some embodiments.

In another example of the present disclosure, a detailed description of the processes by which a first juice 305 of the present disclosure may be generated is found in U.S. Pat. No. 8,679,352 (i.e., the '352 Patent), which is incorporated herein by reference in its entirety as set forth in full. As disclosed in the '352 Patent, a method of processing a microcrop comprising one or more protein separation steps (e.g., centrifugation, precipitation, coagulation) may result in a protein concentrate and a liquor (e.g., liquor, wash-liquor). In accordance with the '352 Patent a protein concentrate may be further processed to generate a dry protein concentrate, while a liquor (e.g., liquor, wash-liquor) may be recycled to growth ponds to be used as a growth medium for a microcrop. A liquor (e.g., liquor, wash-liquor) generated from a method of processing a microcrop disclosed in the '352 Patent may comprise a first juice 305 of the instant disclosure, in some embodiments.

In another example of the present disclosure, a detailed description of the processes by which a first juice 305 may be generated is found in U.S. patent application Ser. No. 15/179,963 (i.e., the '963 Application), which is incorporated herein by reference in its entirety as set forth in full. In accordance with the '963 Application, an extraction of protein and carbohydrate rich products from a microcrop may comprise one or more separation steps, wherein a juice (e.g., juice fraction, first juice, first juice, third juice) from a lysed microcrop may be separated from a solid (e.g., solid fraction, first solid, first cake, second cake). A solid (e.g., solid fraction, first solid, first cake, second cake) may be further processed to generate more juice or a carbohydrate rich product, while a juice (e.g., juice fraction, first juice, first juice, third juice) may be further processed (e.g., by one or more filtrations) to generate a protein rich product.

A juice (e.g., juice fraction, first juice, first juice, third juice) generated from a process for extracting protein and carbohydrate rich products from a microcrop disclosed in the '963 Application may comprise a first juice 305 of the instant disclosure, in some embodiments.

A first juice of the present disclosure may further comprise a reject stream (i.e., reject stream, first reject stream, second reject stream, permeate) from a process for extracting protein and carbohydrate rich products from a microcrop as described in the '963 Application. In accordance with the '963 Application an extraction of protein and carbohydrate rich products from a microcrop may comprise one or more filtration steps, wherein a juice containing a high protein concentration may be filtered to produce a soluble protein product and a reject stream (e.g., reject stream, first reject stream, second reject stream, permeate). The '963 Application describes that a soluble protein product may then be further processed to produce a protein rich product, while a reject stream (e.g., reject stream, first reject stream, second reject stream, permeate) may be either recycled back into a bioreactor system, recycled into a wash solution, or further processed to generate a reject stream product. A reject stream (e.g. reject stream, first reject stream, second reject stream, permeate) of the '963 Application may comprise a first juice 305 of the instant disclosure, in some embodiments.

In another embodiment of the present disclosure, a detailed description of the processes by which a first juice may be generated is found in U.S. patent application Ser. No. 15/751,826 (i.e., the '826 Application), which is incorporated herein by reference in its entirety as set forth in full. In accordance with the '826 Application an extraction of a reduced oxalic acid protein from a microcrop may comprise one or more separation steps, wherein a juice (e.g., juice fraction, first juice, first juice, third juice) from a lysed microcrop may be separated from a solid (e.g., solid fraction, first solid, first cake, second cake). A solid (e.g., solid fraction, first solid, first cake, second cake) may be further processed to generate more juice or a carbohydrate rich product, while a juice (e.g., juice fraction, first juice, first juice, second juice) may be further processed (e.g., by one or more filtrations, to generate a protein rich product). A juice (e.g., juice fraction, first juice, first juice, third juice) from a process for extracting a reduced oxalic acid protein from a microcrop of the '826 Application may comprise a first juice 305 of the instant disclosure, in some embodiments. A first juice 305 of the present disclosure may further comprise a reject stream (e.g., reject stream, first reject stream, second reject stream, permeate) from a process for extracting a reduced oxalic acid protein from a microcrop as described in the '826 Application. In accordance with the '826 Application, an extraction of a reduced oxalic acid protein from a microcrop may comprise one or more filtration steps, wherein a juice containing a high protein concentration may be filtered to produce a soluble protein product and a reject stream (e.g., reject stream, first reject stream, second reject stream, permeate). A soluble protein product may then be further processed to produce a protein rich product, while a reject stream (e.g., reject stream, first reject stream, second reject stream, permeate) may be either recycled back into a bioreactor system, recycled into a wash solution, or further processed to generate a reject stream product. A reject stream (e.g. reject stream, first reject stream, second reject stream, permeate) of the '826 Application may comprise a first juice 305 of the instant disclosure, according to some embodiments. In another example of the present disclosure, a detailed description of the processes by which a first juice may be generated is found in U.S. patent application Ser. No. 15/263,253 (the '253 Application), which is incorporated herein by reference in its entirety as set forth in full. As disclosed in the '253 Application processing of a high concentration protein product from a microcrop may comprise a blanching step, wherein a harvested microcrop is blanched, generating a wet protein concentrate and a blanching solution (e.g., blanching solution, filtered blanching solution, blanching waste). A wet protein concentrate may be further processed to produce a protein concentrate flour, while a blanching solution (e.g., blanching solution, filtered blanching solution, blanching waste) may be further filtered or recycled to an earlier step in the process. A blanching solution (e.g., blanching solution, filtered blanching solution, blanching waste) generated by a process described in the '253 Application for generating a high concentration protein product from a microcrop may comprise a first juice 305 of the instant disclosure.

Additionally, a harvested microcrop may be preliminarily processed (e.g., washing to remove contaminant, soaking to reduce oxalic acid content) without deviating from the scope of the present disclosure. For example, in some embodiments, a harvested microcrop may undergo a wash procedure 101 to remove excess growth medium, debris, contaminants, microorganisms, and/or toxins. As another example, a harvested microcrop may be soaked or buffered in a solution having an adjusted ion content as described in U.S. patent application Ser. No. 15/263,253, which is incorporated herein by reference in its entirety as set forth in full, without deviating from the present disclosure.

In some embodiments a harvested microcrop may be lysed 102 to form a lysed biomass. A detailed description of processes by which a microcrop may be lysed can be found in U.S. patent application Ser. Nos. 13,050,931, 15/179,963, 15/263310, and 15/751,826, each of which is incorporated herein by reference in its entirety as set forth in full. In some embodiments one or more of water (e.g., reverse osmosis water 205), calcium salt 206, and an antioxidant 207 (e.g., sodium metabisulfite) may be added to a microcrop prior to or during a lysing process. The addition of water to a lysed biomass may enhance solubility of protein components. In some embodiments an alkali (e.g., calcium hydroxide, sodium hydroxide) may be added to a microcrop prior to or during a lysing process. The addition of an alkali to a biomass (e.g., lysed biomass) may enhance solubility of protein components.

According to some embodiments, at least some soluble oxalic acid may be removed from a lysed biomass by converting the oxalic acid to an oxalate (e.g., calcium oxalate) and precipitating the oxalate from the lysed biomass (e.g., lysed biomass 301). In some embodiments, precipitating an oxalate from a lysed biomass may include mixing at least a portion of the lysed biomass with at least one calcium salt (e.g., calcium chloride, calcium acetate, calcium hydroxide). Precipitating an oxalate from a lysed biomass, in some embodiments, may include mixing at least a portion of the lysed biomass with a calcium chloride, calcium acetate, or calcium hydroxide solution. Precipitated insoluble oxalate may be removed from the liquid by centrifugation and/or filtration, according to some embodiments.

One or more antioxidants (e.g., sodium metabisulfite, rosemary extract) may be added to a microcrop prior to or during a lysing process to reduce oxidation of the lysed biomass and downstream products. A person having skill in the art would understand that one or more antioxidants may be added to various stages of the disclosed process resulting in a decreased level of oxidation of the disclosed products (e.g., green fiber product 304, colorless protein product 311, and green food product 315) and sub-products (e.g., microcrop milk 313) without deviation from the present disclosure.

According to some embodiments, a lysed biomass may be hydrolyzed 103. Hydrolysis 103 may include adding one or more non-proteolytic enzymes (e.g., pectinase, cellulase, amylase) to the lysed biomass and incubating the mixture at a selected temperature for a selected period of time. A variety of non-proteolytic enzymes may be selected without deviation from the present disclosure. For example, enzymes may be selected for their ability to degrade carbohydrates, fats, fiber, pectin, and other non-protein/peptide cellular materials. An incubation temperature and incubation time may be selected to maximize the effectiveness of the non-proteolytic enzyme(s) selected while minimizing protein degradation. For example, in one embodiment, one or more non-proteolytic enzymes may be added to a lysed biomass and the mixture may be incubated at approximately 50° C. for about 3 hours.

In some embodiments, a pH of a lysed biomass may be adjusted to improve hydrolysis. An adjustment of pH during hydrolysis 103 may be followed later with a corresponding opposite pH adjustment downstream of hydrolysis 103. In some embodiments, a hydrolyzed lysed biomass may be heat treated to deactivate the non-proteolytic enzymes used for hydrolysis 103 (e.g., enzyme combination 208, endogenous microcrop enzymes released during lysis).

Hydrolysis 103 of a lysed biomass may result in improved protein yields in downstream products (e.g., green fiber product 304, microcrop milk 313, colorless protein product 311, and green food product 315) when compared to products derived from a lysed biomass that was not hydrolyzed 103.

### Systems and Methods of Generating a Juice and a Green Protein/Fiber Product

A lysed biomass (e.g., a hydrolyzed lysed biomass) may be separated 104 to generate a juice fraction 302 and a solid fraction 303. A juice fraction may include a protein-rich liquid and/or at least about some solid particles (e.g., carbohydrates, fiber). In some embodiments a lysed biomass may be diluted with a dilution fluid (e.g., water, recycled water, reverse osmosis water) prior to separation. A detailed description of processes by which a lysed biomass (e.g., a hydrolyzed lysed biomass) can be separated to generate a juice fraction and a solid fraction can be found in U.S. Pat. Nos. 13,050,931, 15/179,963, 15/263310, and 15/751,826, each of which is incorporated herein by reference in its entirety as set forth in full.

A juice fraction 302 may be separated to generate a first juice 305 and a first cake 306, according to some embodiments. A first juice may include a dissolved protein. A detailed description of processes by a juice fraction may be separated to generate a first juice and a first cake can be found in U.S. Pat. Nos. 13,050,931, 15/179,963, 15/263310, and 15/751,826, each of which is incorporated herein by reference in its entirety as set forth in full.

A solid fraction 303 may be further separated to extract additional juice (e.g., a second juice). Separation of a solid fraction may form a second juice 307 and a first solid 308. A second juice may include a protein-rich liquid and/or at least some solid particles (e.g., carbohydrates, fiber). A detailed description of processes by which a solid fraction may be separated to form a second juice and a first solid can be found in U.S. Pat. Nos. 13,050,931, 15/179,963, 15/263310, and 15/751,826, each of which is incorporated herein by reference in its entirety as set forth in full.

According to some embodiments, a process for growing, harvesting, and separating a microcrop (e.g., aquatic plant species, Lemna, algal species) may be single cycle and at least one of a first cake 306 and a second cake 309 which are collected at other stages in the cycle (e.g., separation of a juice fraction yields a first cake) may be combined with a first solid to form a solid mixture, and the solid mixture may be further processed.

In some embodiments a process for growing, harvesting, and separating a microcrop (e.g., aquatic plant species, Lemna, algal species) may be multiple cycles or a continuous process such that one or more of a first cake and a second cake that are collected in an earlier cycle may be combined with a solid fraction from a subsequent cycle prior to separation of the solid fraction.

Increasing the extraction of a second juice from a solid fraction may decrease the overall moisture content of a first solid and may thereby lower the energy expenditure required to further process the first solid (e.g., energy required to dry). Additionally, increasing the extraction of juice from a solid fraction and/or solid mixture may improve the yield of a protein-rich product.

In some embodiments, further processing of a first cake 306 and a second juice 307 may be performed. Such additional processing may increase product yield and/or quality. In some embodiments, a first cake and a second juice may be combined and further separated 105 to form a third juice (e.g., third juice 310) and a second cake (e.g., second cake 309). A first cake and a second juice may be independently subjected to further separation, according to some embodiments. A detailed description of processes by which a first cake, a second cake, or any combination thereof may be separated to form a third juice and a second cake can be found in U.S. Pat. Nos. 13,050,931, 15/179,963, 15/263310, and 15/751,826, each of which is incorporated herein by reference in its entirety as set forth in full.

### Green Fiber Product

Some embodiments relate to a process for production of a green fiber product 304 from a biomass of a harvested microcrop (e.g., aquatic plant species, Lemna, algal species). A process may be configured or performed to achieve any desired protein content (e.g., minimal yield, a selected yield). In some embodiments, a protein concentration of a green fiber product 304 is at most about 5%, or at most about 10%, or at most about 15%, or at most about 20%, or at most about 25%, or at most about 30%, or at most about 35% by dry mass basis (DMB) of a green fiber product 304. A remainder of a green fiber product 304 may include carbohydrates, fiber, fats, minerals, or any combination thereof. A green fiber product 304 may be suitable for animal feed and/or human consumption. For example, a green fiber product 304 may serve as an effective replacement for carbohydrate concentrates or isolates (e.g., corn, soy, pea, whey) which are presently used in a large number of animal and human food products either individually or as ingredients and additives.

In some embodiments, a green fiber product 304 may have a fat content lower than about 1%, or lower than about 2%, or lower than about 3%, or lower than about 4%, or lower than about 5%, or lower than about 6%, or lower than about 7%, or lower than about 8%, or lower than about 9% by DMB of the green fiber product 304. According to some embodiments, a green fiber product 304 may include an ash content consisting of a residue containing inorganic mineral elements. An ash content in some embodiments may be determined by combusting a green fiber product 304 at a high temperature (e.g., ≥500° C.) to remove organic matter. A green fiber product 304 may have an ash content lower than about 2%, or lower than about 4%, or lower than about 6%, or lower than about 8%, or lower than about 10%, or lower than about 12%, or lower than about 13%, or lower than about 1% to about 15% by DMB of the green fiber product 304 in some embodiments. A green fiber product 304 may be further processed to meet a desired ash content (e.g., higher or lower concentration, a desired ash composition).

According to some embodiments, a green fiber product 304 may have a carbohydrate content higher than about 30%, or higher than about 40%, or higher than about 50%, or higher than about 60%, or higher than about 70%, or higher than about 75%, or higher than about 80% by DMB of the green fiber product 304. A green fiber product 304, in some embodiments, may have a carbohydrate content from about 30% to about 40%, or from about 40% to about 50%, or from about 50% to about 60%, or from about 60% to about 70%, or from about 70% to about 80% by DMB of the green fiber product 304. In some embodiments, a green fiber product 304 may have a carbohydrate content from about 1% to about 50%, or from about 2% to about 60%, or from about 5% to about 70%, or from about 8% to about 80% by DMB of the green fiber product 304. A green fiber product 304 may be further processed to meet a desired carbohydrate content (e.g., higher or lower concentration, a desired carbohydrate composition). In some embodiments, green fiber products may have a composition that is between about 10% and 25% protein, about 5% and 15% ash, about 2% and 6% fat, and about 55% and 70% carbohydrates and other materials. For example, green fiber products A, B, C, and D produced by the processes described herein may include the contents summarized in Table 2 below.

TABLE 2

| | Green fiber | Green fiber | Green fiber | Green fiber |
|---|---|---|---|---|
| Composition | product A | product B | product C | product D |
| Protein | 20% | 22.5% | 18% | 25% |
| Ash | 7% | 8% | 10% | 8% |
| Fat | 3% | 4% | 6% | 5% |
| Carbs/Other | 70% | 65.5% | 66% | 62% |

### Systems and Methods of Generating a Hydrolyzed Protein Juice

### Generation of a Protein Suspension

In some embodiments a juice (e.g., first juice 305) may be treated 110 (e.g., heating, pasteurization, heating to specific temperatures and maintaining said temperature for a specific time, high pressure homogenization), to generate a protein suspension 311. Treatment 110 may inactivate or kill some or all microorganisms (e.g., bacteria, fungi, viruses) that may be present in a juice (e.g., first juice 305). It is desirable to decrease a population of microorganisms present in a juice as microorganisms can be pathogenic (e.g., Salmonella, E. coli, Listeria) and/or can contribute to spoilage of the juice (e.g., decreased usable lifespan). Treatment 110 of a juice may decrease or eliminate microorganisms from a juice thereby improving safety for human consumption and prolonging the shelf life of products generated using the juice. In some embodiments, treatment 110 may deactivate enzymes (e.g., endogenous microcrop enzymes, microbial enzymes) present in a juice. Enzymes can contribute to spoilage of a juice or contribute undesirable characteristics (e.g., flavors, colors) to downstream products; therefore, inactivating such enzymes can be desirable. Treatment 110 of a juice may result in an improved taste, odor, and color in downstream products compared to products generated from a juice that was not subjected to the treatment 110.

Treatment 110 may be performed using any method (e.g., pasteurization, heating to specific temperatures and maintaining said temperature for a specific time, high pressure homogenization) suitable for decreasing a microbial population or enzymatic load of a juice to a desired level without resulting in unacceptable levels of degradation to a soluble protein present in a juice. In some embodiments treatment 110 may result in denaturation of soluble protein (e.g., by heat) and flocculation. Such flocculation may be counteracted by a downstream hydrolysis treatment 112 where insoluble proteins are converted to soluble protein hydrolysates.

Treatment 110 may comprise heating (e.g., rapidly, slowly) a juice (e.g., first juice 305) to a specific temperature (e.g., 70° C., 80° C.) and maintaining the juice at the temperature for a sufficient period of time (e.g., 15 sec-1 min) to achieve the desired amount of microbial deactivation and/or enzymatic deactivation. For example, treatment 110 may include pasteurization. However, treatment 110 may also include processes which would not qualify as pasteurization per se due to the rapidity of heating, temperatures selected, or time retained at the designated temperature but which are still capable of achieving a sufficient amount of microbial deactivation and/or enzymatic deactivation to provide improved qualities to downstream products (e.g., expanded shelf life, improved flavor, reduced spoilage). Treatment 110 may include sequentially heating a juice to multiple temperatures with each temperature being held for a designated period of time where such periods of time may be the same or different from one another. A selected temperature may include one or more temperatures sufficient to destroy and/or inactivate microorganisms (e.g., pathogenic bacteria, yeast, mold, Salmonella, E. coli, Listeria) and/or enzymes. A selected temperature may include a temperature of about 5° C., or about 7° C., or about 10° C., or about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C., or about 85° C., or about 90° C. A juice may be held at the selected temperature for a sufficient period of time to achieve the desired amount of microbial deactivation and/or enzymatic deactivation. In some embodiments, treatment 110 may be carried out using at least a steam injection heat exchanger.

Treatment 110 may include exposing a juice (e.g., first juice 305) to beta and/or gamma rays for a sufficient period of time to achieve the desired amount of microbial deactivation and/or enzymatic deactivation. Treatment 110, according to some embodiments, may include heating a juice (e.g., first juice 305) to a suitable temperature (e.g., incubation temperature) for hydrolysis 112. For example, a juice (e.g., first juice 305) may be heated to about 20° C., or about 25° C., or about 30° C., or about 35° C., or about 40° C., or about 45° C., or about 50° C., or about 55° C., or about 60° C.

A protein suspension 311 may include both soluble and denatured protein. A protein suspension 311 may comprise a juice having soluble proteins and other soluble, plant derived compounds together with insoluble complexes and aggregates generated by treatment 110. Insoluble complexes and aggregates may include of protein, chlorophyll and other compounds.

### Cooling a Protein Suspension

In some embodiments a protein suspension 311 generated by treatment 110 of a juice (e.g., first juice 305) may be cooled 111 (e.g., rapidly) to a cooled temperature. A cooled temperature may be a suitable temperature (e.g., incubation temperature) for hydrolysis 112.

For example, a juice (e.g., first juice 305) may be cooled to about 20° C., or about 25° C., or about 30° C., or about 35° C., or about 40° C., or about 45° C., or about 50° C., or about 55° C., or about 60° C.

Any number of cooling techniques may be used to reduce a temperature of a protein suspension. For example, in some embodiments heat exchange mechanisms may be used to cool a protein suspension.

### Hydrolysis of a Protein Suspension

A juice (e.g. first juice 305) or a protein suspension 311 (e.g., cooled, uncooled) may be hydrolyzed to generate a hydrolyzed protein juice 313. A hydrolyzed protein juice 313 may include peptides, amino acids, and other soluble phytonutrients such ash, minerals and a suspension/solution of high molecular weight protein/pigment complexes.

Hydrolysis 112 may include adding one or more proteolytic enzymes (e.g., protease, peptidase, subtilisin, aminopeptidase, dipeptidyl peptidase, endopeptidases, etc.tc.) to the juice or the protein suspension to generate a mixture and incubating the mixture at a selected temperature for a selected period of time to generate a hydrolyzed protein juice 313. A variety of proteolytic enzymes may be selected without deviation from the present disclosure. For example, enzymes may be selected for their ability to achieve varying degrees of hydrolysis of proteins and peptides, or cleave specific peptide bonds using endopeptidases and exopeptidases. An incubation temperature and incubation time may be selected to maximize the effectiveness of the proteolytic enzyme(s) selected. In some embodiments an incubation temperature may be about 20° C., or about 25° C., or about 30° C., or about 35° C., or about 40° C., or about 45° C., or about 50° C., or about 55° C., or about 60° C., or about 65° C., or about 70° C., or about 75° C., or about 80° C., or about 85° C., or about 90° C., or about 95° C. An incubation time may range from about 30 min to about 4 hours, in some embodiments. For example, in one embodiment one or more proteolytic enzymes may be added to a juice (e.g., first juice 305) or a protein suspension 311 (e.g., cooled, uncooled) and the mixture may be incubated at approximately 50° C. for about 3 hours to generate a hydrolyzed protein juice 313. In another embodiment, one or more proteolytic enzymes (e.g., subtilisin A) may be added to a juice (e.g., first juice 305) or a protein suspension 311 (e.g., cooled, uncooled) and the mixture may be incubated at approximately 60° C. for about 1 hour to generate a hydrolyzed protein juice 313. Hydrolysis may be performed in batch and/or continuous stir tank configurations, according to some embodiments.

In some embodiments one or more antioxidants may be added to juice or a protein suspension prior to hydrolysis 112 to reduce oxidation of the hydrolyzed protein juice 313 and downstream products (e.g., colorless protein product 311, microcrop milk 313, and green food product 315). In some embodiments, a pH of a mixture may be adjusted to improve hydrolysis. An adjustment of pH during hydrolysis 112 may be followed later with a corresponding opposite pH adjustment a hydrolyzed protein juice 313. In some embodiments, a hydrolyzed protein juice 313 may be heat treated to deactivate the proteolytic enzymes used for hydrolysis. For example, a hydrolyzed protein juice may be heated to about 65° C., or to about 70° C., or to about 75° C., or to about 80° C., or to about 85° C., or to about 90° C. Deactivation (e.g., heat treatment) time may range from about 3 minutes to about 20 minutes. For example, a hydrolyzed protein juice may be heated to about 80° C. for 10 minutes to deactivate the one or more proteolytic enzymes. A person having skill in the art would understand that the precise temperature and time combination may be adjusted depending upon the specific enzymes selected.

In some embodiments, proteolytic enzymes selection and incubation temperatures may be selected to achieve a hydrolyzed protein juice 313 having specific characteristics (e.g., specific degree of hydrolysis, specific distribution of molecular weight hydrolysates including proteins, peptides, and amino acids). For example, in some embodiments a specific degree of hydrolysis may be selected to achieve selected odor, taste, or color profiles of an end product.

In some embodiments, proteolytic enzymes selection and incubation temperatures may be selected to achieve downstream products (e.g., colorless protein product 311, microcrop milk 313, and green food product 315) having specific characteristics (e.g., solubility, foaming, gelling, emulsification, water absorption, fat holding, flavor).

According to some embodiments, methods of hydrolysis 112 described herein may separate bound protein material from green thylakoid material in a protein suspension 311. Accordingly, a hydrolyzed protein juice 313 may be more easily processed to produce products which are green colored (e.g., green food product) and products which are not green colored (e.g., colorless protein product 311, microcrop milk 313, electrolyte drink 322), which may appeal to some consumers.

### Systems and Methods of Processing a Hydrolyzed Protein Juice

Filtration and/ Separation of a Hydrolyzed Protein Juice

According to some implementations, hydrolysis 112 may be combined with a filtration process (e.g., filtration 113) in processing a protein suspension (e.g., protein suspension 311) to generate a protein concentrate (e.g., high yield protein concentrate) which is further processed into a product (e.g., colorless protein product 311 and microcrop milk 313) downstream. In some embodiments, following a hydrolysis 112 process with a filtration 113 process advantageously enables a high yielding extraction and separation of protein that may be natively bound with pigments from a microcrop. In some instances, using hydrolysis 112 and filtration 113 to extract and separate protein natively bound with pigments requires no use of solvents (e.g., expensive organic/inorganic solvents or the like) and other capital equipment thus reducing production costs.

In some embodiments, a hydrolyzed protein juice 313 undergoes filtration 113 to separate high molecular weight green protein/pigment complexes (e.g., chlorophyll and chlorophyll complexes) from low molecular weight hydrolyzed protein peptides and amino acids (e.g., reduced color, colorless, non-green). Filtration 113, according to some embodiments, may include at least one of microfiltration and ultrafiltration to generate a green retentate 314 and a decolored permeate 316. Filtration 113 may include one or more of microfiltration and ultrafiltration of a hydrolyzed protein juice 313.

Microfiltration, ultrafiltration, or a combination thereof may be used to separate suspended solids including high molecular weight green protein/pigment complexes (e.g., chlorophyll and chlorophyll complexes) and other cellular components (e.g., fats, fiber, cellular debris) from liquid and soluble components of a hydrolyzed protein juice 313. In some embodiments, a hydrolyzed protein juice 313 may be filtered using microfiltration, ultrafiltration, or a combination thereof to generate a permeate 316 (e.g., non-green permeate or decolored permeate) and a green retentate 314. Suitable filter sizes for microfiltration may include, in some embodiments, ≤about 1 μm, or ≤about 0.5 μm, or ≤about 0.4 μm, or ≤about 0.3 μm, or ≤about 0.2 μm, or ≤about 0.1 μm, or ≤about 500 kilodalton (kDa). According to some implementations, the filter size may be a design parameter that depends on a selectable balance between protein yield and protein color desired in products (e.g., colorless protein product 311, microcrop milk 313, and green food product 315) downstream. In some cases, any protein resulting from hydrolysis 112 and/or filtration 113 may be routed/rerouted to a given product downstream as necessary to prevent any waste. For example, although not shown in the figures, any excess protein after generating a given product (e.g., colorless protein product 311) may be routed back and added to another product (e.g., green food product 315) being produced.

A person having skill in the art would understand that the selection of filter sizing used for filtration 113 may be adjusted according to the desired product specifications. For example, a slightly larger filter size (e.g., 1 μm) may be acceptable where it would be acceptable for the desired downstream products from the decolored permeate (e.g., colorless protein product 311, microcrop milk 313, and green food product 315) to have some chlorophyll content (e.g., chlorophyll and chlorophyll complexes) resulting in some green coloration of the product. Similarly, a smaller filter size (e.g., 500 kDa, 0.1 μm) may be acceptable where the desired downstream products from the permeate (e.g., colorless protein product 311, microcrop milk 313, and green food product 315) have little to no chlorophyll content (e.g., chlorophyll and chlorophyll complexes) and no discernible green coloration of the product; however, the use of smaller filter sizes may reduce yield of downstream products from the permeate. A person having skill in the art would understand the relationship between these trade-offs and select appropriate filter sizes for the desired results (e.g., product characteristics, yield).

In some embodiments a hydrolyzed protein juice 307 may be separated without a need for filtration 113. A hydrolyzed protein juice 307 may be separated into a liquid stream and a solid stream using centrifugation, for example a high-speed disc stack centrifuge or a decanter centrifuge. The liquid stream may then be further processed (e.g., demineralization 116, etc.) while the solid stream may be processed by dewatering 119 to ultimately result in a green food product 303.

In some embodiments a hydrolyzed protein juice 307 may be separated prior to filtration 113. A hydrolyzed protein juice 307 may be separated into a liquid stream and a solid stream using centrifugation, for example a high-speed disc stack centrifuge or a decanter centrifuge. The liquid stream may then be further processed by filtration 113 while the solid stream may be processed by dewatering 119 to ultimately result in a green food product 303.

### Systems and Methods of Generating a Green Food Product

In some embodiments a green retentate 314 may be further processed to generate a green food product 315.

### Green Food Product

Some embodiments relate to a process for production of a green food product 315 from a biomass of a harvested microcrop (e.g., aquatic plant species, Lemna, algal species).

A green food product 315 may have a high concentration of chlorophyll, nutritionally beneficial fats, and other pigments and pigment complexes. A process may be configured or performed to achieve any desired protein content (e.g., minimal yield, a selected yield) in a green food product. In some embodiments, a protein concentration of a green food product 315 is at most about 25%, or at most about 30%, or at most about 35%, or at most 40%, or at most about 45%, or at most about 50%, or at most about 55%, or at most about 60%, or at most about 70% by dry mass basis (DMB) of a green food product 315. A remainder of a green food product 315 may include carbohydrates, fiber, fats, minerals, or any combination thereof. A green food product 315 may be suitable for animal feed and/or human consumption. For example, a green food product 315 may serve as an effective nutritional supplement (e.g., chlorophyll, vitamins, antioxidants, zeaxanthins, omega 3, phytonutrients, and probiotics/prebiotics) which are presently used in a large number of human food products either individually or as ingredients and additives.

In some embodiments, a green food product 315 may have a fat content up to about 18%, or up to about 22%, or up to about 25%, or up to about 28%, or up to about 30%, or up to about 32%, or up to about 34%, or up to about 35%, or up to about 37%, or up to about 39%, or up to about 41%, or up to about 43% by DMB of the green food product 315. A green food product 315 may have a fat content from about 12% to about 28%, or from about 16% to about 30%, or from about 18% to about 34%, or from about 20% to about 35%, or from about 22% to about 38%, or from about 24% to about 39%, or from about 25% to about 40%, or from about 26% to about 41%, or from about 27% to about 42%, or from about 28% to about 43%, or from about 17% to about 40% or from about 19% to about 33% by DMB of the green food product 315 in some embodiments. A green food product 315 may be further processed to meet a desired fat content (e.g., higher or lower concentration, a desired fat composition).

According to some embodiments, a green food product 315 may include an ash content consisting of a residue containing inorganic mineral elements. An ash content in some embodiments may be determined by combusting a green food product 315 at a high temperature (e.g., ≥500° C.) to remove organic matter. A green food product 315 may have an ash content lower than about 2%, or lower than about 4%, or lower than about 6%, or lower than about 8%, or lower than about 10%, or lower than about 12%, or lower than about 13%, or lower than about 1% to about 15% by DMB of the green food product 315 in some embodiments. A green food product 315 may be further processed to meet a desired ash content (e.g., higher or lower concentration, a desired ash composition).

According to some embodiments, a green food product 315 may have a carbohydrate content lower than about 3%, or lower than about 5%, or lower than about 8%, or lower than about 10%, or lower than about 12%, or lower than about 14%, or lower than about 15%, or lower than about 16% by DMB of the green food product. A green food product 315, in some embodiments, may have a carbohydrate content from about 1% to about 8%, or from about 2% to about 10%, or from about 3% to about 11%, or from about 4% to about 12%, or from about 2% to about 13.4%, or from about 1% to about 15% by DMB of the green food product 315. A green food product 315 may be further processed to meet a desired carbohydrate content (e.g., higher or lower concentration, a desired carbohydrate composition). In one embodiment, little to no fiber is bound to the green food product 315.

According to some embodiments, a green food product 315 may include Vitamin B12. In some embodiments, a green food product 315 may include one or more bioactive forms of Vitamin B12 (e.g., Adenosylcobalmin, Hydroxycobalmin, and Methylcobalmin). A concentration of Vitamin B12 may range from about 0.1 ug/100 g (DWB) to about 200 ug/100 g. For example, a concentration of bioactive Vitamin B12 may be between about 0.1 to about 0.5 ug/100 g, or about 0.5 to about 1 ug/100 g, or about 1 to about 5 ug/100 g, or about 5 to about 10 ug/100 g, or about 10 to about 20 ug/100 g, or about 20 to about 30 ug/100 g, or about 30 to about 40 ug/100 g, or about 40 to about 50 ug/100 g, or about 50 to about 60 ug/100 g, or about 60 to about 70 ug/100 g, or about 70 to about 80 ug/100 g, or about 80 to about 90 ug/100 g, or about 90 to about 100 ug/100 g, or about 100 to about 110 ug/100 g, or about 110 to about 120 ug/100 g, or about 120 to about 130 ug/100 g, or about 130 to about 140 ug/100 g, or about 140 to about 150 ug/100 g, or about 150 to about 160 ug/100 g, or about 160 to about 170 ug/100 g, or about 170 to about 180 ug/100 g, or about 180 to about 190 ug/100 g, or about 190 to about 200 ug/100 g. In some embodiments, a Vitamin B12 content may range from about 0.0000001% to about 0.0000005% (DWB), or about 0.0000005% to about 0.000001%, or about 0.000001% to about 0.000005%, or about 0.000005% to about 0.00001%, or about 0.00001% to about 0.00002%, or about 0.00002% to about 0.00003%, or about 0.00003% to about 0.00004%, or about 0.00004% to about 0.00005%, or about 0.00005% to about 0.00006%, or about 0.00006% to about 0.00007%, or about 0.00007% to about 0.00008%, or about 0.00008% to about 0.00009%, or about 0.00009% to about 0.0001%, or about 0.0001% to about 0.00011%, or about 0.00011% to about 0.00012%, or about 0.00012% to about 0.00013%, or about 0.00013% to about 0.00014%, or about 0.00014% to about 0.00015%, or about 0.00015% to about 0.00016%, or about 0.00016% to about 0.00017%, or about 0.00017% to about 0.00018%, or about 0.00018% to about 0.00019%, or about 0.00019% to about 0.0002%.

In some embodiments, green food products may have a composition that is between about 42 and 54% protein, about 3 and 9% ash, about 20 and 32% fat, about 8 and 20% carbohydrates and other materials, and contains between about 3 and 30 ug/100 g Vitamin B12. For example, green fiber products A, B, C, and D produced by the processes described herein may include the contents summarized in Table 3 below. For example, green food products A, B, C, and D produced by the processes described herein may include the contents summarized in Table 3 below.

TABLE 3

| | Green food | Green food | Green food | Green food |
|---|---|---|---|---|
| Composition | product A | product B | product C | product D |
| Protein | 50% | 52.5% | 48.8% | 54% |
| Ash | 9% | 8% | 9% | 6% |
| Fat | 28% | 30% | 32% | 28% |
| Carbs/Other | 13% | 9.5% | 10.2% | 12% |
| Vitamin B12 | 18 ug/100 g | 5 ug/100 g | 22 ug/100 g | 30 ug/100 g |

### Dewatering

In some embodiments a dewatering process 119 may be used to reduce a moisture content of a green retentate 314. Reducing a moisture content of a green retentate 314 may reduce capital and operational expenditures, for example, by reducing the energy needed to dry an end product (e.g., green food product 315). In some embodiments a dewatering process 119 may be performed using reverse osmosis, forward osmosis, nanofiltration, evaporation, vacuum belt, or any combination thereof.

According to some embodiments, suitable filter sizes for reverse osmosis filtration may include ≤about 0.001 μm, ≤about 0.0009 μm, ≤about 0.0008 μm, ≤about 0.0007 μm, ≤about 0.0006 μm, ≤about 0.0005 μm, ≤about 0.0004 μm, ≤about 0.0003 μm, ≤about 0.0002 μm, or ≤about 0.0001 μm. A reverse osmosis filter may have a filter size of not more than about 0.001 μm, in some embodiments.

In some embodiments, suitable filter sizes for nanofiltration may include ≤about 0.01 μm, or ≤about 0.009 μm, or ≤about 0.008 μm, or ≤about 0.007 μm, or ≤about 0.006 μm, or ≤about 0.005 μm, or ≤about 0.004 μm, or ≤about 0.003 μm, or ≤about 0.002 μm, or ≤about 0.001 μm.

In some embodiments an evaporation process may be used to reduce a moisture content of a green retentate 314. Evaporation may be performed by, for example, a thermal (evaporative) means such as: a rising film evaporator, a falling film evaporator, a natural circulation evaporator (vertical or horizontal), an agitated-film evaporator, a multiple-effect evaporator, by vacuum evaporation, or any combination thereof. Heat may be supplied directly into the evaporator, or indirectly through a heat jacket. Heat may either come from a raw source (e.g., combustion of natural gas, steam from a boiler) or from a waste heat stream (e.g., dryer exhaust) or from heat transferred by cooling an input stream.

According to some embodiments, a moisture content of a green retentate 314 may be reduced using gravity separation, draining, an inclined screen, a vibratory screen, filtration, a decanter centrifuge, a belt press, a fan press, a rotary press, a screw press, a filter press, a finisher press, or any combination thereof.

According to some implementations, a dewatering process may involve little to no heat in order to specific quality aspects of downstream products (e.g., prevent oxidation, degradation).

### Treatment of Dewatered Green Retentate

In some embodiments an antioxidant (e.g., rosemary extract, sodium metabisulfite, Eugenol) may be mixed with a green retentate 314 prior to drying to, for example, reduce oxidation of fatty acids (e.g., omega 3) and improve shelf life of products (e.g., shelf life of a packaged product) generated using the dewatered green retentate 314 (e.g., a green food product 315).

A treatment 124 may be performed prior to drying to, for example, decrease a microbial population or enzymatic load of a green retentate 314. As described above, a treatment 124 may be performed using any method (e.g., pasteurization, heating to specific temperatures and maintaining said temperature for a specific time, gamma ray exposure, treating with high pressure homogenization) suitable for decreasing a microbial population or enzymatic load of a juice to a desired level without resulting in unacceptable levels of degradation to a soluble protein present in a green retentate 314.

In some embodiments, a treatment 124 of a dewatered green retentate 314 may be similar to or the same as a treatment 110 of a first juice 305 described above.

### Drying

A green retentate 314 (e.g., dewatered green retentate, treated dewatered green retentate) may be dried to generate a green food product 315. A drying procedure 120, in some embodiments, may reduce the moisture content of a green retentate 314 (e.g., dewatered green retentate) to a desired level (e.g., lower moisture content, a desired moisture content). A moisture content of a green food product 315 may be, for example, below about 95%, or below about 90%, or below about 80%, or below about 70%, or below about 60%, or below about 50%, or below about 40%, or below about 30%, or below about 20%, or below about 10%, or below about 5%, or below about 1% by weight of the green food product 315, in some embodiments. A drying procedure 120 may be performed using a mechanism including, for example, a spray dryer (preferred), a drum dryer, a double drum dryer, flash dryer, a fluid-bed dryer, a convection dryer, an evaporator, or any combination thereof.

In some embodiments, an inlet temperature of a dryer mechanism (the temperature at the entrance to a dryer) may be above 25° C., or above 50° C., or above 75° C., or above 100° C., or above 125° C., or above 150° C., or above 175° C., or above 200° C., or above 225° C., or above 250° C., or above 275° C., or above 300° C., or above 325° C., or above 350° C., or above 375° C., or above 400° C., or above 425° C., or above 450° C., or above 475° C., or above 500° C. An inlet temperature, in some embodiments, may be from about 25° C. to about 50° C., or from about 50° C. to about 75° C., or from about 75° C. to about 100° C., or from about 100° C. to about 125° C., or from about 125° C. to about 150° C., or from about 150° C. to about 175° C., or from about 175° C. to about 200° C., or from about 200° C. to about 225° C., or from about 225° C. to about 250° C., or from about 250° C. to about 275° C., or from about 275° C. to about 300° C., or from about 300° C. to about 325° C., or from about 325° C. to about 350° C., or from about 350° C. to about 375° C., or from about 375° C. to about 400° C., or from about 400° C. to about 425° C., or from about 425° C. to about 450° C., or from about 450° C. to about 475° C., or from about 475° C. to about 500° C., or above 500° C. An inlet temperature may be from about 50° C. to about 100° C., or from about 100° C. to about 150° C., or from about 150° C. to about 200° C., or from about 200° C. to about 250° C., or from about 250° C. to about 300° C., or from about 300° C. to about 350° C., or from about 350° C. to about 400° C., or from about 400° C. to about 450° C., or from about 450° C. to about 500° C., or above 500° C., in some embodiments. According to some embodiments, an inlet temperature of a dryer mechanism may be about 225° C.

According to some embodiments, an outlet temperature of a dryer mechanism (the temperature at the exit from a dryer) may be below about 300° C., or below about 275° C., or below about 250° C., or below about 225° C., or below about 200° C., or below about 175° C., or below about 150° C., or below about 125° C., or below about 100° C., or below about 75° C., or below about 50° C., or below about 25° C. An outlet temperature may be from about 300° C. to about 275° C., or from about 275° C. to about 250° C., or from about 250° C. to about 225° C., or from about 225° C. to about 200° C., or from about 200° C. to about 175° C., or from about 175° C. to about 150° C., or from about 150° C. to about 125° C., or from about 125° C. to about 100 ° C., or from about 100° C. to about 75° C., or from about 75° C. to about 50° C., or from about 50° C. to about 25° C., or below about 25° C., in some embodiments. An outlet temperature, in some embodiments, may be from about 300° C. to about 250° C., or from about 250° C. to about 200° C., or from about 200° C. to about 150° C., or from about 150° C. to about 100° C., from about 100° C. to about 50° C., or from about 50° C. to about 25° C., or below about 25° C. According to some embodiments, an outlet temperature of a dryer mechanism may be about 75° C.

In some embodiments, a volume of a green retentate 314 (e.g., a dewatered green retentate) may be mixed with a volume of a green food product 303 having a lower moisture content than the green retentate 314 prior to drying. This process, known as back-mixing, may be employed when, for example, the moisture content of a green retentate 314 exceeds the level that a dryer mechanism is capable of accepting. By back-mixing a green food product 303 having a lower moisture content with a green retentate 314, a total moisture content may be kept within the specifications of a dryer mechanism, thereby reducing operational costs (e.g., wear and tear on equipment).

According to some embodiments, a green food product 315 may be milled to form a green flour. A milling procedure may involve jet mill, a shear mill, a hammer mill, a pin mill, a vibrating mill, a fluid energy mill, or any combination thereof.

An antioxidant (e.g., rosemary extract, sodium metabisulfite) may be mixed with a green food product 303 before packaging, according to some embodiments.

According to some embodiments, a green retentate 314 (e.g., dewatered) may be frozen, flash-frozen, or freeze dried. In some embodiments, a green retentate 314 (e.g., dewatered) may be milled prior to drying.

### Systems and Methods of Generating a Colorless Protein Product

In some embodiments a decolored permeate 316 resulting from filtration 113 may be further processed to generate a colorless protein product 311.

### Colorless Protein Product

Some embodiments relate to a process for production of a colorless protein product 311 from a biomass of a harvested microcrop (e.g., aquatic plant species, Lemna, algal species). A process may be configured or performed to achieve any desired protein yield (e.g., maximal yield, a selected yield). In some embodiments, a protein concentration of a colorless protein product 311 is higher than about 30%, or higher than about 40%, or higher than about 45%, or higher than about 50%, or higher than about 55%, or higher than about 60%, or higher than about 65%, or higher than about 70%, or higher than about 75% by dry mass basis (DMB) of a colorless protein product 311. A remainder of a colorless protein product 311 may include carbohydrates, fiber, fats, minerals, other phytonutrients, or any combination thereof. A colorless protein product 311 may be suitable for animal feed and/or human consumption. For example, a colorless protein product 311 may serve as an effective replacement for protein concentrates or isolates (e.g., soy, pea, whey) which are presently used in a large number of human food products either individually or as ingredients and additives. According to some embodiments, at least of portion of a protein composition of a colorless protein product 311 may comprise denatured or partially-denatured protein, peptides, and/or amino acids. In one embodiment, a colorless protein product 311 may be generated without the use of organic and/or inorganic solvents.

In some embodiments, a colorless protein product 311 may have a fat content lower than about 1%, or lower than about 2%, or lower than about 3%, or lower than about 4%, or lower than about 5%, or lower than about 6%, or lower than about 7% by DMB of the colorless protein product 311. A colorless protein product 311 may have a fat content from about 1% to about 7% by DMB of the colorless protein product 311 in some embodiments. A colorless protein product 311 may be further processed to meet a desired fat content (e.g., higher or lower concentration, a desired fat composition).

According to some embodiments, a colorless protein product 311 may include an ash content consisting of a residue containing inorganic mineral elements. An ash content in some embodiments may be determined by combusting a colorless protein product 311 at a high temperature (e.g., ≥500° C.) to remove organic matter. A colorless protein product 311 may have an ash content lower than about 2%, or lower than about 4%, or lower than about 6%, or lower than about 8%, or lower than about 10%, or lower than about 12%, or lower than about 15%, or lower than about 20% by DMB of the colorless protein product 311 in some embodiments. A colorless protein product 311 may be further processed to meet a desired ash content (e.g., higher or lower concentration, a desired ash composition).

According to some embodiments, a colorless protein product 311 may have a carbohydrate content lower than about 8%, or lower than about 10%, or lower than about 14%, or lower than about 16%, or lower than about 18%, or lower than about 20% by DMB of the colorless protein product. A colorless protein product 311, in some embodiments, may have a carbohydrate content from about 4% to about 18%, or from about 3% to about 16%, or from about 2% to about 12%, or from about 5% to about 20% by DMB of the colorless protein product 311. In some embodiments, a colorless protein product 311 may have a carbohydrate content from about 1% to about 20%, or from about 2% to about 22%, or from about 5% to about 18%, or from about 8% to about 16% by DMB of the colorless protein product 311. A colorless protein product 311 may be further processed to meet a desired carbohydrate content (e.g., higher or lower concentration, a desired carbohydrate composition).

According to some embodiments, a colorless protein product 311 may include Vitamin B12. In some embodiments, a colorless protein product 311 may include one or more bioactive forms of Vitamin B12 (e.g., Adenosylcobalmin, Hydroxycobalmin, and Methylcobalmin) A concentration of Vitamin B12 may range from about 0.1 ug/100 g (DWB) to about 200 ug/100 g. For example, a concentration of bioactive Vitamin B12 may be between about 0.1 to about 0.5 ug/100 g, or about 0.5 to about 1 ug/100 g, or about 1 to about 5 ug/100 g, or about 5 to about 10 ug/100 g, or about 10 to about 20 ug/100 g, or about 20 to about 30 ug/100 g, or about 30 to about 40 ug/100 g, or about 40 to about 50 ug/100 g, or about 50 to about 60 ug/100 g, or about 60 to about 70 ug/100 g, or about 70 to about 80 ug/100 g, or about 80 to about 90 ug/100 g, or about 90 to about 100 ug/100 g, or about 100 to about 110 ug/100 g, or about 110 to about 120 ug/100 g, or about 120 to about 130 ug/100 g, or about 130 to about 140 ug/100 g, or about 140 to about 150 ug/100 g, or about 150 to about 160 ug/100 g, or about 160 to about 170 ug/100 g, or about 170 to about 180 ug/100 g, or about 180 to about 190 ug/100 g, or about 190 to about 200 ug/100 g. In some embodiments, a Vitamin B12 content may range from about 0.0000001% to about 0.0000005% (DWB), or about 0.0000005% to about 0.000001%, or about 0.000001% to about 0.000005%, or about 0.000005% to about 0.00001%, or about 0.00001% to about 0.00002%, or about 0.00002% to about 0.00003%, or about 0.00003% to about 0.00004%, or about 0.00004% to about 0.00005%, or about 0.00005% to about 0.00006%, or about 0.00006% to about 0.00007%, or about 0.00007% to about 0.00008%, or about 0.00008% to about 0.00009%, or about 0.00009% to about 0.0001%, or about 0.0001% to about 0.00011%, or about 0.00011% to about 0.00012%, or about 0.00012% to about 0.00013%, or about 0.00013% to about 0.00014%, or about 0.00014% to about 0.00015%, or about 0.00015% to about 0.00016%, or about 0.00016% to about 0.00017%, or about 0.00017% to about 0.00018%, or about 0.00018% to about 0.00019%, or about 0.00019% to about 0.0002%.

In some embodiments, colorless protein products may have a composition that is between about 55 and 75% protein, about 9 and 20% ash, about 0 and 3% fat, about 5 and 15% carbohydrates and other materials, and contains between about 3 and 30 ug/100 g Vitamin B12. For example, a colorless protein product 311 produced by the processes described herein may include the contents summarized in Table 4 below.

TABLE 4

| | Colorless | Colorless | Colorless | Colorless |
|---|---|---|---|---|
| | Protein | Protein | Protein | Protein |
| Composition | product A | product B | product C | product D |
| Protein | 68% | 75% | 68.7% | 71.2% |
| Ash | 18% | 9% | 14% | 12.5% |
| Fat | 2% | 2.5% | 3% | 2.8% |
| Carbs/Other | 12% | 13.5% | 14.3% | 13.5% |
| Vitamin B12 | 22 ug/100 g | 18 ug/100 g | 20 ug/100 g | 10 ug/100 g |

### Adsorption

In some embodiments a decolored permeate 316 generated by filtration 113 may include one or more polyphenols which can contribute undesirable color (e.g., darken) to a decolored permeate 316 and/or result in undesirable flavors in downstream products (e.g., colorless protein product 311 and microcrop milk 313). Accordingly, in some embodiments it may be desirable to remove polyphenols from a decolored permeate 316 using an adsorption process 114. In some implementations, the removal of polyphenols from a decolored permeate 316 may be carried out in a reactor vessel.

An adsorption process 114 may be implemented using one or more polymers configured to bind polyphenols. According to one embodiment, an adsorption process 114 may include mixing a polymer (e.g., a hydrogen bonding polymer, polyvinylpolypyrrolidone (PVPP)) 211 with a volume of a decolored permeate 316. For example, in some embodiments a polymer 211 may be mixed with a decolored permeate 316 at a ratio of 0.1 g of polymer/L of decolored permeate, or 0.5 g/L, or 0.8 g/L, or 1 g/L, or 1.5 g/L, or 2 g/L, or 2.5 g/L, or 3 g/L, or 3.5 g/L, or 4 g/L, or 4.5 g/L, or 5 g/L. According to some embodiments, an adsorption process 114 may include continuously mixing a volume of a polymer with a volume of a decolored permeate for a specified period of time (e.g., 20 min, 1 hour, 2 hours). Specific ratios, volumes, and mixing times may be specific to the polymer selected and other parameters (e.g., amount of polyphenol content).

In some embodiments, an adsorption process 114 may include passing a decolored permeate 316 through one or more hydrogen bonding resins and/or ion exchange resins. In some embodiments, an adsorption process 114 may include passing a decolored permeate 316 through a series (e.g., at least two, at least three) of hydrogen bonding resins and/or ion exchange resins. Each hydrogen bonding resins and/or ion exchange resin in a series may be the same or different than the other hydrogen bonding resins and/or ion exchange resins in the series. In some embodiments, a hydrogen bonding resin may comprise polyvinylpolypyrrolidone (PVPP). In some embodiments an ion exchange resin may be a strongly acidic resin, a strongly basic resin, a weakly acidic resin, a weakly basic resin, a weak anion exchange resin, a strong anion exchange resin, a weak cation exchange resin, a strong cation exchange resin, or any combination thereof. Appropriate anionic exchange resins may include, but are not limited to, those anion exchange resins which include a trialkyl ammonium salt. A trialkyl ammonium salt may include a functional group including a halide, an alkyl group selected from C1-C16 alkyl, an aryl, a branched chain alkyl, and a cycloalkyl. A halide may include one or more of a fluoride, a chloride, a bromide, and an iodide. According to some embodiments, an anion exchange resin may include a trialkyl ammonium salt having three methyl groups and a chloride, thereby forming a trimethylammonium chloride salt.

Hydrogen bonding and/or ion exchange resins may be used in a batch mode or arranged in a continuous process, whereby resins may be cycled through adsorption 114 and regeneration processes. In some embodiments adsorption 114 may further comprise adjusting a pH of a decolored permeate 316 or a product yielded from an ion exchange column (e.g., polyphenol reduced decolored permeate).

A person having skill in the art would understand that alternative methods for removing one or more polyphenols from a colorless permeate may be implemented without deviating from the scope of this disclosure.

### Clarification

In some embodiments, an adsorption 114 process may result in a decolored permeate 316 having a reduced polyphenol content, but retaining one or more polymers used for the adsorption. Accordingly, a polymer may be removed from a decolored permeate 316 having a reduced polyphenol content using a clarification 115 process. In some embodiments, clarification 115 may include any number of methods appropriate for separating a non-soluble polymer from a decolored permeate 316 having a reduced polyphenol content including gravitational separation, precipitation, filtering, and centrifugation According to some implementations, a clarification process 115 may take place in a settling tank or a centrifuge.

A person having skill in the art would understand that alternative methods for separating a non-soluble polymer from a decolored permeate having a reduced polyphenol content 309 may be implemented without deviating from the scope of this disclosure.

### Demineralization

A decolored permeate 316 (e.g., a decolored permeate having a reduced polyphenol content 317) may be further processed by demineralization 116 to generate a demineralized stream 320 and a mineral stream 318. Demineralization 116 may remove one or more undesirable components (e.g., ash, minerals, organic compounds) from a decolored permeate 316 (e.g., a decolored permeate having a reduced polyphenol content 317) to generate a demineralized stream 320 having a reduced content of the undesirable component.

According to some embodiments, a demineralized stream 320 may have a lower mineral content in comparison to an intake fluid that was treated to a demineralization 116. A demineralized stream 320 may have a mineral content from about 1% to about 90% of a mineral content contained in an intake fluid that was treated to a demineralization 116. For example, a demineralized stream 320 may have a mineral content of about 1%, or of about 10%, or of about 20%, or of about 30%, or of about 40%, or of about 50%, or of about 60%, or of about 70%, or of about 80%, or of about 90%, or about 95% of a mineral content contained in an intake fluid that was treated to a demineralization 116, where about includes plus or minus 5%. In some embodiments, a demineralized stream 320 contains a soluble protein.

Ionic components that may be removed from an intake fluid include lithium, sodium, potassium, calcium, magnesium, phosphorus, ammonium, chloride, fluoride, bromide, iodide, sulfate, nitrate, nitrite, salts thereof, and combinations thereof. In some embodiments, a demineralization process 116 may produce a demineralized stream 320 that has from about 1% to about 90% of an ionic component content of an intake fluid. For example, a resulting a demineralized stream 320 may contain about 1% of an ionic component, or about 10% of an ionic component, or about 20% of an ionic component, or about 30% of an ionic component, or about 40% of an ionic component, or about 50% of an ionic component, or about 60% of an ionic component, or about 70% of an ionic component, or about 80% of an ionic component, or about 90% of an ionic component, in comparison to an intake fluid, where about includes plus or minus 5%.

In some embodiments, a mineral stream may be recycled to a bioreactor, may undergo a mixing procedure 121, and combinations thereof.

Demineralization 116 may include one or more of treating a decolored permeate 316 (e.g., a decolored permeate having a reduced polyphenol content 317) with electrodialysis, nanofiltration, diafiltration, and the use of hydrogen bonding and/or ion exchange resins.

In some embodiments, demineralization 116 may include electrodialysis of a decolored permeate 316. Electrodialysis may involve passing the decolored permeate 316 through an electrodialysis cell to generate a demineralized stream 320 and a mineral stream 318. In some embodiments an electrodialysis cell may contain: a positively charged anode, a negatively charged cathode, an anionic selective membrane, a cationic selective membrane, an ion-depleted cell (for the collection of demineralized stream 320), and an ion-concentrated cell (for collection of a mineral stream 318).

Demineralization 116 may include nanofiltration of a decolored permeate 316. In some embodiments, suitable filter sizes for nanofiltration may include ≤about 1000 Da, or ≤about 900 Da, or ≤about 800 Da, or ≤about 700 Da, or ≤about 600 Da, or ≤about 500 Da, or ≤about 400 Da, or ≤about 300 Da, or ≤about 200 Da, or ≤about 100 Da.

### Diafiltration

According to some embodiments, demineralization 116 may be used to reduce an ionic component (e.g., mineral) component of an intake fluid including a decolored permeate 316.

In some embodiments, to remove salts and minerals from an intake fluid, diafiltration may be used to produce a demineralized stream 320 and a mineral stream 318. Diafiltration may include passing an intake fluid through a semi-permeable membrane to create a demineralized stream 320 containing a soluble protein and a mineral stream 318 containing a mineral. A demineralized stream 320 may be collected separately or may be subsequently undergo a dewatering process 117, a mixing procedure 121, and combinations thereof. A mineral stream 318 may include calcium, sodium, lithium, sodium, potassium, calcium, magnesium, phosphorus, ammonium, chloride, fluoride, bromide, iodide, sulfate, zinc, chromium, selenium, iron, copper, aluminum, silicon, aluminum, nitrate, nitrite, salts thereof, and combinations thereof.

In some embodiments, a diafiltration process may produce a demineralized stream 320 that has from about 1% to about 90% of a mineral component content of an intake fluid. For example, a resulting a demineralized stream 320 may contain about 1% of a mineral component, or about 10% of the mineral component, or about 20% of the mineral component, or about 30% of the mineral component, or about 40% of the mineral component, or about 50% of the mineral component, or about 60% of the mineral component, or about 70% of the mineral component, or about 80% of the mineral component, or about 90% of the mineral component, in comparison to an intake fluid, where about includes plus or minus 5%.

According to some embodiments, demineralization 116 may include passing a decolored permeate 316 (e.g., a decolored permeate having a reduced polyphenol content 317) through one or more hydrogen bonding and/or ion exchange resins. In some embodiments, demineralization 116 may comprise passing a decolored permeate 316 (e.g., a decolored permeate having a reduced polyphenol content 317) through a series (e.g., at least two, at least three) of hydrogen bonding and/or ion exchange resins. Each hydrogen bonding and/or ion exchange resin in a series may be the same or different than the other hydrogen bonding and/or ion exchange resins in the series. In some embodiments, a hydrogen bonding resin may comprise polyvinylpolypyrrolidone (PVPP). In some embodiments an ion exchange resin may be a strongly acidic resin, a strongly basic resin, a weakly acidic resin, a weakly basic resin, a weak anion exchange resin, a strong anion exchange resin, a weak cation exchange resin, a strong cation exchange resin, or any combination thereof. Appropriate anionic exchange resins may include, but are not limited to, those anion exchange resins which include a trialkyl ammonium salt. A trialkyl ammonium salt may include a functional group including a halide, an alkyl group selected from C1-C16 alkyl, an aryl, a branched chain alkyl, and a cycloalkyl. A halide may include one or more of a fluoride, a chloride, a bromide, and an iodide. According to some embodiments, an anion exchange resin may include a trialkyl ammonium salt having three methyl groups and a chloride, thereby forming a trimethylammonium chloride salt.

Hydrogen bonding and/or ion exchange resins may be used in a batch mode or arranged in a continuous process, whereby resins may be cycled through demineralization and regeneration processes. In some embodiments demineralization 116 may further comprise adjusting a pH of a decolored permeate 316 (e.g., a decolored permeate having a reduced polyphenol content 317) or a mineral stream 318.

### Dewatering

In some embodiments a dewatering process 117 may be used to reduce a moisture content of a demineralized stream 320. Reducing a moisture content of a demineralized stream 320 may reduce capital and operational expenditures, for example, by reducing the energy needed to dry an end protein product (e.g., colorless protein product 302).

In some embodiments a dewatering process 119 may be performed using reverse osmosis, nanofiltration, evaporation, or any combination thereof.

According to some embodiments, suitable filter sizes for reverse osmosis filtration may include ≤about 0.001 μm, ≤about 0.0009 μm, ≤about 0.0008 μm, ≤about 0.0007 μm, ≤about 0.0006 μm, ≤about 0.0005 μm, ≤about 0.0004 μm, ≤about 0.0003 μm, ≤about 0.0002 μm, or ≤about 0.0001 μm. A reverse osmosis filter may have a filter size of not more than about 0.001 μm, in some embodiments.

In some embodiments, suitable filter sizes for nanofiltration may include ≤about 0.01 μ, or ≤about 0.009 μm, or ≤about 0.008 μm, or ≤about 0.007 μm, or ≤about 0.006 μm, or ≤about 0.005 μm, or ≤about 0.004 μm, or ≤about 0.003 μm, or ≤about 0.002 μm, or ≤about 0.001 μm.

In some embodiments an evaporation process may be used to reduce a moisture content of a demineralized stream 320. Evaporation may be performed by, for example, a thermal (evaporative) means such as: a rising film evaporator, a falling film evaporator, a natural circulation evaporator (vertical or horizontal), an agitated-film evaporator, a multiple-effect evaporator, by vacuum evaporation, or any combination thereof. Heat may be supplied directly into the evaporator, or indirectly through a heat jacket. Heat may either come from a raw source (e.g., combustion of natural gas, steam from a boiler) or from a waste heat stream (e.g., dryer exhaust) or from heat transferred by cooling the input stream.

According to some embodiments, a moisture content of a demineralized stream 320 may be reduced using gravity separation, draining, an inclined screen, a vibratory screen, filtration, a decanter centrifuge, a belt press, a fan press, a rotary press, a screw press, a filter press, a finisher press, or any combination thereof.

### Treatment of Demineralized Stream

In some embodiments an antioxidant (e.g., rosemary extract, sodium metabisulfite) may be mixed with a demineralized stream 320 prior to drying to improve shelf life of products (e.g., shelf life of a packaged product) generated using the dewatered demineralized stream (e.g., a colorless protein product 311).

A treatment 125 may be performed prior to drying to, for example, decrease a microbial population or enzymatic load of a demineralized stream 320. As described above, a treatment 125 may be performed using any method (e.g., pasteurization, heating to specific temperatures and maintaining said temperature for a specific time, gamma ray exposure) suitable for decreasing a microbial population or enzymatic load of a demineralized stream 320 to a desired level without resulting in unacceptable levels of degradation to a soluble protein present in a demineralized stream 320.

In some embodiments, a treatment 125 of a demineralized stream 320 may be similar to or the same as a treatment 110 of a first juice 305 described above.

### Drying

A demineralized stream 320 (e.g., dewatered) may be dried 117 to generate a colorless protein product 311. A drying procedure 117, in some embodiments, may reduce the moisture content of a demineralized stream 320 (e.g., dewatered demineralized stream) to a desired level (e.g., lower moisture content, a desired moisture content). A moisture content of a colorless protein product 311 may be, for example, below about 95%, or below about 90%, or below about 80%, or below about 70%, or below about 60%, or below about 50%, or below about 40%, or below about 30%, or below about 20%, or below about 10%, or below about 5%, or below about 1% by weight of the a colorless protein product 311, in some embodiments. A drying procedure 117 may be performed using a mechanism including, for example, a spray dryer (preferred), a drum dryer, a double drum dryer, flash dryer, a fluid-bed dryer, a convection dryer, an evaporator, or any combination thereof.

In some embodiments, an inlet temperature of a dryer mechanism (the temperature at the entrance to a dryer) may be above 25° C., or above 50° C., or above 75° C., or above 100° C., or above 125° C., or above 150° C., or above 175° C., or above 200° C., or above 225° C., or above 250° C., or above 275° C., or above 300° C., or above 325° C., or above 350° C., or above 375° C., or above 400° C., or above 425° C., or above 450° C., or above 475° C., or above 500°

C. An inlet temperature, in some embodiments, may be from about 25° C. to about 50° C., or from about 50° C. to about 75° C., or from about 75° C. to about 100° C., or from about 100° C. to about 125° C., or from about 125° C. to about 150° C., or from about 150° C. to about 175° C., or from about 175° C. to about 200° C., or from about 200° C. to about 225° C., or from about 225° C. to about 250° C., or from about 250° C. to about 275° C., or from about 275° C. to about 300° C., or from about 300° C. to about 325° C., or from about 325° C. to about 350° C., or from about 350° C. to about 375° C., or from about 375° C. to about 400° C., or from about 400° C. to about 425° C., or from about 425° C. to about 450° C., or from about 450° C. to about 475° C., or from about 475° C. to about 500° C., or above 500° C. An inlet temperature may be from about 50° C. to about 100° C., or from about 100° C. to about 150° C., or from about 150° C. to about 200° C., or from about 200° C. to about 250° C., or from about 250° C. to about 300° C., or from about 300° C. to about 350° C., or from about 350° C. to about 400° C., or from about 400° C. to about 450° C., or from about 450° C. to about 500° C., or above 500° C., in some embodiments. According to some embodiments, an inlet temperature of a dryer mechanism may be about 225° C.

According to some embodiments, an outlet temperature of a dryer mechanism (the temperature at the exit from a dryer) may be below about 300° C., or below about 275° C., or below about 250° C., or below about 225° C., or below about 200° C., or below about 175° C., or below about 150° C., or below about 125° C., or below about 100° C., or below about 75° C., or below about 50° C., or below about 25° C. An outlet temperature may be from about 300°

C. to about 275° C., or from about 275° C. to about 250° C., or from about 250° C. to about 225° C., or from about 225° C. to about 200° C., or from about 200° C. to about 175° C., or from about 175° C. to about 150° C., or from about 150° C. to about 125° C., or from about 125° C. to about 100 ° C., or from about 100° C. to about 75° C., or from about 75° C. to about 50° C., or from about 50° C. to about 25° C., or below about 25° C., in some embodiments. An outlet temperature, in some embodiments, may be from about 300° C. to about 250° C., or from about 250° C. to about 200° C., or from about 200° C. to about 150° C., or from about 150° C. to about 100° C., from about 100° C. to about 50° C., or from about 50° C. to about 25° C., or below about 25° C. According to some embodiments, an outlet temperature of a dryer mechanism may be about 75° C.

In some embodiments, a volume of a demineralized stream 320 (e.g., dewatered demineralized stream) may be mixed with a volume of a colorless protein product 311 having a lower moisture content than the demineralized stream 320 prior to drying. This process, known as back-mixing, may be employed when, for example, the moisture content of a demineralized stream 320 (e.g., dewatered demineralized stream) exceeds the level that a dryer mechanism is capable of accepting. By back-mixing a colorless protein product 311 having a lower moisture content with a demineralized stream 320, a total moisture content may be kept within the specifications of a dryer mechanism, thereby reducing operational costs (e.g., wear and tear on equipment).

According to some embodiments, a colorless protein product 311 may be milled to form a colorless protein flour. A milling procedure may involve a jet mill, a shear mill, a hammer mill, a pin mill, a vibrating mill, a fluid energy mill, or any combination thereof. An antioxidant (e.g., rosemary extract, sodium metabisulfite) may be mixed with a colorless protein product 311 before packaging, according to some embodiments.

According to some embodiments, a demineralized stream 320 (e.g., dewatered) may be frozen, flash-frozen, or freeze dried. In some embodiments, a demineralized stream 320 (e.g., dewatered) may be milled prior to drying.

### Colorimetric Measurement

According to some embodiments, a method of generating a colorless protein product may include a colorimetric measurement. In some embodiments, a colorimetric measurement may be based on International Commission on Illumination's L*a*b* (CIELAB) color space coordinates, wherein color is measured as three values: L* for the lightness from black (0) to white (100), a* from green (−) to red (+), and b* from blue (−) to yellow (+). Any measurement with an a* value >−1 is not perceived to be green. For example, a colorless protein product may have an a* value >−1.

Systems and Methods of Generating a Microcrop Milk, a Dried Base Powder, and/or an Electrolyte Drink

The present disclosure further relates to methods of producing a microcrop milk 313, a dried base powder 324, and/or an electrolyte drink 322. A microcrop milk 313, a dried base powder 324, and/or an electrolyte drink 322 may be produced by: (1) mixing 121 one or more of a mineral stream 318, a demineralized stream 320, and a dewatered demineralized stream to generate a milk mixture 319; (2) dewatering 122 a milk mixture 319 to generate a milk base 312; (3) formulating a milk base 312 to generate a microcrop milk 313; and (4) drying one or more of a milk mixture 319 and a milk base 312 to generate a dried base powder 324.

### Microcrop Milk

Some embodiments relate to a process for production of a microcrop milk 313 from a biomass of a harvested microcrop (e.g., aquatic plant species, Lemna, algal species). A process may be configured or performed to achieve any desired protein yield (e.g., maximal yield, a selected yield) of a microcrop milk 313. In some embodiments, a protein concentration of a microcrop milk 313 is higher than about 10%, or higher than about 20%, or higher than about 30%, or higher than about 40%, or higher than about 45%, or higher than about 50%, or higher than about 55%, or higher than about 60%, or higher than about 65%, or higher than about 70% by dry mass basis (DMB) of a microcrop milk 313. A remainder of a microcrop milk 313 may include carbohydrates, fiber, fats, minerals, or any combination thereof.

In some embodiments, a microcrop milk 313 may have a fat content from about 10% to about 40% by DMB of the microcrop milk. A microcrop milk 313 may have a fat content of at least 10%, or at least 15%, or at least 20%, or at least 25%, or at least 30%, or at least 35%, or at least 40% by DMB of the microcrop milk in some embodiments.

According to some embodiments, a microcrop milk 313 may include an ash content consisting of a residue containing inorganic mineral elements. An ash content in some embodiments may be determined by combusting a microcrop milk 313 at a high temperature (e.g., ≥500° C.) to remove organic matter. A microcrop milk 313 may have an ash content lower than about 2%, or lower than about 4%, or lower than about 6%, or lower than about 8%, or lower than about 10%, or lower than about 12%, or lower than about 13%, or lower than about 1% to about 15% by DMB of the microcrop milk 313 in some embodiments.

According to some embodiments, a microcrop milk 313 may have a carbohydrate of between about 10% and about 55% by DMB of the microcrop milk. A microcrop milk 313 may have a carbohydrate content of about 10%, or about 15%, or about 20%, or about 25%, or about 30%, or about 35%, or about 40%, or about 45%, or about 50%, or about 55% by DMB of the microcrop milk, in some embodiments.

According to some embodiments, a microcrop milk 313 may include Vitamin B12. In some embodiments, a microcrop milk 313 may include one or more bioactive forms of Vitamin B12 (e.g., Adenosylcobalmin, Hydroxycobalmin, and Methylcobalmin). A concentration of Vitamin B12 may range from about 0.1 ug/100 g (DWB) to about 200 ug/100 g. For example, a concentration of bioactive Vitamin B12 may be between about 0.1 to about 0.5 ug/100 g, or about 0.5 to about 1 ug/100 g, or about 1 to about 5 ug/100 g, or about 5 to about 10 ug/100 g, or about 10 to about 20 ug/100 g, or about 20 to about 30 ug/100 g, or about 30 to about 40 ug/100 g, or about 40 to about 50 ug/100 g, or about 50 to about 60 ug/100 g, or about 60 to about 70 ug/100 g, or about 70 to about 80 ug/100 g, or about 80 to about 90 ug/100 g, or about 90 to about 100 ug/100 g, or about 100 to about 110 ug/100 g, or about 110 to about 120 ug/100 g, or about 120 to about 130 ug/100 g, or about 130 to about 140 ug/100 g, or about 140 to about 150 ug/100 g, or about 150 to about 160 ug/100 g, or about 160 to about 170 ug/100 g, or about 170 to about 180 ug/100 g, or about 180 to about 190 ug/100 g, or about 190 to about 200 ug/100 g. In some embodiments, a Vitamin B12 content may range from about 0.0000001% to about 0.0000005% (DWB), or about 0.0000005% to about 0.000001%, or about 0.000001% to about 0.000005%, or about 0.000005% to about 0.00001%, or about 0.00001% to about 0.00002%, or about 0.00002% to about 0.00003%, or about 0.00003% to about 0.00004%, or about 0.00004% to about 0.00005%, or about 0.00005% to about 0.00006%, or about 0.00006% to about 0.00007%, or about 0.00007% to about 0.00008%, or about 0.00008% to about 0.00009%, or about 0.00009% to about 0.0001%, or about 0.0001% to about 0.00011%, or about 0.00011% to about 0.00012%, or about 0.00012% to about 0.00013%, or about 0.00013% to about 0.00014%, or about 0.00014% to about 0.00015%, or about 0.00015% to about 0.00016%, or about 0.00016% to about 0.00017%, or about 0.00017% to about 0.00018%, or about 0.00018% to about 0.00019%, or about 0.00019% to about 0.0002%.

In some embodiments, a microcrop milk may have a composition that is between about 30 and 55% protein, about 5 and 15% ash, about 20 and 36% fat, about 10 and 45% carbohydrates and other materials, and contains between about 3 and 30 ug/100 g Vitamin B12. For example, a microcrop milk 313 produced by the processes described herein may include the contents summarized in Table 5 below.

TABLE 5

| | Microcrop | Microcrop | Microcrop | Microcrop | Microcrop |
|---|---|---|---|---|---|
| Composition | Milk A | Milk B | Milk C | Milk D | Milk E |
| Protein | 32% | 54% | 46% | 37% | 43% |
| Ash | 5% | 5% | 15% | 10% | 5% |
| Fat | 20% | 26% | 22% | 21% | 24% |
| Carbs/ | 43% | 15% | 17% | 32% | 28% |
| Other | | | | | |
| Vitamin | 18 ug/ | 10 ug/ | 22 ug/ | 20 ug/ | 30 ug/ |
| B12 | 100 g | 100 g | 100 g | 100 g | 100 g |

### Electrolyte Drink

Some embodiments relate to a process for production of an electrolyte drink 322 from a biomass of a harvested microcrop (e.g., aquatic plant species, Lemna, algal species). In these embodiments, one or more processes may be optimized to achieve an appropriate blend and concentration of nutritional components, flavor, mouthfeel, color, and/or to maximize mineral content without the constraints on ionic strength which may be present when generating a microcrop milk 313. A process may be configured or performed to achieve any desired protein yield (e.g., maximal yield, a selected yield) of an electrolyte drink 322. In some embodiments, a protein concentration of an electrolyte drink 322 is higher than about 10%, or higher than about 20%, or higher than about 30%, or higher than about 40%, or higher than about 45%, or higher than about 50%, or higher than about 55%, or higher than about 60%, or higher than about 65%, or higher than about 70% by dry mass basis (DMB) of an electrolyte drink 322. A remainder of an electrolyte drink 322 may include carbohydrates, fiber, fats, minerals, or any combination thereof.

In some embodiments, an electrolyte drink 322 may have a fat content from about 0% to about 40% by DMB of the microcrop milk. An electrolyte drink 322 may have a fat content of between 0% and 10%, or at least 10%, or at least 15%, or at least 20%, or at least 25%, or at least 30%, or at least 35%, or at least 40% by DMB of the microcrop milk in some embodiments.

According to some embodiments, an electrolyte drink 322 may include an ash content consisting of a residue containing inorganic mineral elements. An ash content in some embodiments may be determined by combusting an electrolyte drink 322 at a high temperature (e.g., ≥500° C.) to remove organic matter. An electrolyte drink 322 may have an ash content lower than about 2%, or lower than about 4%, or lower than about 6%, or lower than about 8%, or lower than about 10%, or lower than about 12%, or lower than about 13%, or lower than about 1% to about 15% by DMB of the electrolyte drink 322 in some embodiments.

According to some embodiments, an electrolyte drink 322 may have a carbohydrate of between about 10% and about 55% by DMB of the electrolyte drink. An electrolyte drink 322 may have a carbohydrate content of about 10%, or about 15%, or about 20%, or about 25%, or about 30%, or about 35%, or about 40%, or about 45%, or about 50%, or about 55% by DMB of the electrolyte drink, in some embodiments.

According to some embodiments, an electrolyte drink 322 may include Vitamin B12. In some embodiments, an electrolyte drink 322 may include one or more bioactive forms of Vitamin B12 (e.g., Adenosylcobalmin, Hydroxycobalmin, and Methylcobalmin). A concentration of Vitamin B12 may range from about 0.1 ug/100 g (DWB) to about 200 ug/100 g. For example, a concentration of bioactive Vitamin B12 may be between about 0.1 to about 0.5 ug/100 g, or about 0.5 to about 1 ug/100 g, or about 1 to about 5 ug/100 g, or about 5 to about 10 ug/100 g, or about 10 to about 20 ug/100 g, or about 20 to about 30 ug/100 g, or about 30 to about 40 ug/100 g, or about 40 to about 50 ug/100 g, or about 50 to about 60 ug/100 g, or about 60 to about 70 ug/100 g, or about 70 to about 80 ug/100 g, or about 80 to about 90 ug/100 g, or about 90 to about 100 ug/100 g, or about 100 to about 110 ug/100 g, or about 110 to about 120 ug/100 g, or about 120 to about 130 ug/100 g, or about 130 to about 140 ug/100 g, or about 140 to about 150 ug/100 g, or about 150 to about 160 ug/100 g, or about 160 to about 170 ug/100 g, or about 170 to about 180 ug/100 g, or about 180 to about 190 ug/100 g, or about 190 to about 200 ug/100 g. In some embodiments, a Vitamin B12 content may range from about 0.0000001% to about 0.0000005% (DWB), or about 0.0000005% to about 0.000001%, or about 0.000001% to about 0.000005%, or about 0.000005% to about 0.00001%, or about 0.00001% to about 0.00002%, or about 0.00002% to about 0.00003%, or about 0.00003% to about 0.00004%, or about 0.00004% to about 0.00005%, or about 0.00005% to about 0.00006%, or about 0.00006% to about 0.00007%, or about 0.00007% to about 0.00008%, or about 0.00008% to about 0.00009%, or about 0.00009% to about 0.0001%, or about 0.0001% to about 0.00011%, or about 0.00011% to about 0.00012%, or about 0.00012% to about 0.00013%, or about 0.00013% to about 0.00014%, or about 0.00014% to about 0.00015%, or about 0.00015% to about 0.00016%, or about 0.00016% to about 0.00017%, or about 0.00017% to about 0.00018%, or about 0.00018% to about 0.00019%, or about 0.00019% to about 0.0002%.

An electrolyte drink 322 may have varying content levels depending on the desired range of flavors and/or nutritional content (e.g., Vitamin B12 levels, protein levels, carbohydrate levels, etc.). In some embodiments, an electrolyte drink may have a composition that is between about 15 and 45% protein, about 15 and 60% ash, about 0 and 10% fat, about 15 and 55% carbohydrates and other materials, and contains between about 3 and 50 ug/100 g Vitamin B12. For example, an electrolyte drink 322 produced by the processes described herein may include, but are not limited by, the contents summarized in Table 6 below.

TABLE 6

| | Electrolyte | Electrolyte | Electrolyte | Electrolyte | Electrolyte |
|---|---|---|---|---|---|
| Composition | Drink A | Drink B | Drink C | Drink D | Drink E |
| Protein | 15% | 30% | 45% | 42.5% | 48% |
| Ash | 29% | 30% | 22.7% | 18% | 36.7% |
| Fat | <1% | <1% | 3% | 3% | <1% |
| Carbs/Other | 55% | 39% | 29.3% | 36.5% | 15.3% |
| Vitamin | 19 ug/ | 9 ug/ | 18 ug/ | 27 ug/ | 30 ug/ |
| B12 | 100 g | 100 g | 100 g | 100 g | 100 g |

### Dried Base Powder

Some embodiments relate to a process for production of a dried base powder 324 from a biomass of a harvested microcrop (e.g., aquatic plant species, Lemna, algal species). A process may be configured or performed to achieve any desired protein yield (e.g., maximal yield, a selected yield) of a dried base powder 324. In some embodiments, a protein concentration of a dried base powder 324 is higher than about 10%, or higher than about 20%, or higher than about 30%, or higher than about 40%, or higher than about 45%, or higher than about 50%, or higher than about 55%, or higher than about 60%, or higher than about 65%, or higher than about 70% by dry mass basis (DMB) of a dried base powder 324. A remainder of a dried base powder 324 may include carbohydrates, fiber, fats, minerals, or any combination thereof.

In some embodiments, a dried base powder 324 may have a fat content from about 0% to about 40% by DMB of the dried base powder 324. A dried base powder 324 may have a fat content of between 0% and 10%, or at least 10%, or at least 15%, or at least 20%, or at least 25%, or at least 30%, or at least 35%, or at least 40% by DMB of the dried base powder in some embodiments.

According to some embodiments, a dried base powder 324 may include an ash content consisting of a residue containing inorganic mineral elements. An ash content in some embodiments may be determined by combusting a dried base powder 324 at a high temperature (e.g., ≥500° C.) to remove organic matter. A dried base powder 324 may have an ash content lower than about 2%, or lower than about 4%, or lower than about 6%, or lower than about 8%, or lower than about 10%, or lower than about 12%, or lower than about 13%, or lower than about 1% to about 15% by DMB of the dried base powder 324 in some embodiments.

According to some embodiments, a dried base powder 324 may have a carbohydrate of between about 10% and about 55% by DMB of the dried base powder 324. A dried base powder 324 may have a carbohydrate content of about 10%, or about 15%, or about 20%, or about 25%, or about 30%, or about 35%, or about 40%, or about 45%, or about 50%, or about 55% by DMB of the dried base powder 324, in some embodiments.

According to some embodiments, a dried base powder 324 may include Vitamin B12. In some embodiments, a dried base powder 324 may include one or more bioactive forms of Vitamin B12 (e.g., Adenosylcobalmin, Hydroxycobalmin, and Methylcobalmin). A concentration of Vitamin B12 may range from about 0.1 ug/100 g (DWB) to about 200 ug/100 g. For example, a concentration of bioactive Vitamin B12 may be between about 0.1 to about 0.5 ug/100 g, or about 0.5 to about 1 ug/100 g, or about 1 to about 5 ug/100 g, or about 5 to about 10 ug/100 g, or about 10 to about 20 ug/100 g, or about 20 to about 30 ug/100 g, or about 30 to about 40 ug/100 g, or about 40 to about 50 ug/100 g, or about 50 to about 60 ug/100 g, or about 60 to about 70 ug/100 g, or about 70 to about 80 ug/100 g, or about 80 to about 90 ug/100 g, or about 90 to about 100 ug/100 g, or about 100 to about 110 ug/100 g, or about 110 to about 120 ug/100 g, or about 120 to about 130 ug/100 g, or about 130 to about 140 ug/100 g, or about 140 to about 150 ug/100 g, or about 150 to about 160 ug/100 g, or about 160 to about 170 ug/100 g, or about 170 to about 180 ug/100 g, or about 180 to about 190 ug/100 g, or about 190 to about 200 ug/100 g. In some embodiments, a Vitamin B12 content may range from about 0.0000001% to about 0.0000005% (DWB), or about 0.0000005% to about 0.000001%, or about 0.000001% to about 0.000005%, or about 0.000005% to about 0.00001%, or about 0.00001% to about 0.00002%, or about 0.00002% to about 0.00003%, or about 0.00003% to about 0.00004%, or about 0.00004% to about 0.00005%, or about 0.00005% to about 0.00006%, or about 0.00006% to about 0.00007%, or about 0.00007% to about 0.00008%, or about 0.00008% to about 0.00009%, or about 0.00009% to about 0.0001%, or about 0.0001% to about 0.00011%, or about 0.00011% to about 0.00012%, or about 0.00012% to about 0.00013%, or about 0.00013% to about 0.00014%, or about 0.00014% to about 0.00015%, or about 0.00015% to about 0.00016%, or about 0.00016% to about 0.00017%, or about 0.00017% to about 0.00018%, or about 0.00018% to about 0.00019%, or about 0.00019% to about 0.0002%.

A dried base powder 324 may have varying content levels depending on the desired range of flavors and/or nutritional content (e.g., Vitamin B12 levels, protein levels, carbohydrate levels, etc.). In some embodiments, a dried base powder may have a composition that is between about 15 and 60% protein, about 10 and 60% ash, about 0 and 10% fat, about 15 and 55% carbohydrates and other materials, and contains between about 3 and 50 ug/100 g Vitamin B12. For example, a dried base powder 324 produced by the processes described herein may include, but are not limited by, the contents summarized in Table 7 below.

TABLE 7

| Com- | Dried Base | Dried Base | Dried Base | Dried Base | Dried Base |
|---|---|---|---|---|---|
| position | Powder A | Powder B | Powder C | Powder D | Powder E |
| Protein | 55% | 50% | 60% | 42.5% | 48% |
| Ash | 13% | 30% | 22.7% | 18% | 16.7% |
| Fat | <1% | <1% | 3% | 3% | <1% |
| Carbs/Other | 31% | 19% | 14.3% | 36.5% | 34.3% |
| Vitamin | 19 ug/ | 9 ug/ | 18 ug/ | 27 ug/ | 30 ug/ |
| B12 | 100 g | 100 g | 100 g | 100 g | 100 g |

### Mixing to Generate a Milk Mixture

A milk mixture 319 may be generated by mixing 121 one or more of a mineral stream 318, a demineralized stream 320, and a dewatered demineralized stream. The relative volumes of each of the mineral stream 318, the demineralized stream 320, and the dewatered demineralized stream mixed to form the milk mixture may be adjusted depending upon the compositions of the relative components (e.g., protein composition of the demineralized stream, ash composition of the mineral permeate) and the desired composition (e.g., protein concentration) of the milk mixture.

Any number of methods may be used to mix the combination of one or more of a mineral stream 318, a demineralized stream 320, and a dewatered demineralized stream including for example, bleeding a fraction of each stream via proportional control valves into a continuous stir tank mixer. Such method may be part of a feedback control system based on one or more measurements of the milk mixture 319.

In some embodiments, a pH value of a milk mixture may be adjusted to a desired level by adding acid or base.

In some embodiments, a milk mixture 319 may be consumed and may constitute a product on its own without further processing.

### Dewatering to Generate a Milk Base

In some embodiments, a milk mixture 319 may have an undesirably high moisture content for its intended purpose and thereby may be dewatered to reduce its volume. According to some embodiments, dewatering 122 may include one or more of nanofiltration, reverse osmosis filtration, and evaporation.

In some embodiments, a dewatering 122 comprises evaporation. Evaporation may be performed by, for example, a thermal (evaporative) means such as: a rising film evaporator, a falling film evaporator, a natural circulation evaporator (vertical or horizontal), an agitated-film evaporator, a multiple-effect evaporator, by vacuum evaporation, or any combination thereof. Heat may be supplied directly into the evaporator, or indirectly through a heat jacket. Heat may either come from a raw source (e.g., combustion of natural gas, steam from a boiler) or from a waste heat stream (e.g., dryer exhaust) or from heat transferred by cooling an input stream. In some embodiments, the heat added during a dewatering 122 step comprising evaporation may effectively pasteurize the concentrated product (e.g., milk base). In some embodiments, concentration by evaporation may comprise more than one heating and cooling cycle.

A dewatering step 122 may comprise a reduction in moisture content by nanofiltration or reverse osmosis filtration. In some embodiments, suitable filter sizes for nanofiltration may include ≤about 0.01 μm, or ≤about 0.009 μm, or ≤about 0.008 μm, or ≤about 0.007 μm, or ≤about 0.006 μm, or ≤about 0.005 μm, or ≤about 0.004 μm, or ≤about 0.003 μm, or ≤about 0.002 μm, or ≤about 0.001 μm. According to some embodiments, suitable filter sizes for reverse osmosis filtration may include ≤about 0.001 μm, ≤about 0.0009 μm, ≤about 0.0008 ≤about 0.0007 μm, ≤about 0.0006 μm, ≤about 0.0005 ≤about 0.0004 μm, ≤about 0.0003 μm, ≤about 0.0002 μm, or ≤about 0.0001 μm.

A milk mixture 319 that has been dewatered 122 may result in a milk base 312 that is any one of a liquid, a granule, and a powder. In some embodiments, a milk base 312 may be consumed and may constitute a product on its own without further processing.

### Drying a Milk Mixture or a Milk Base to Generate a Dried Base Powder

A milk base 312 and/or a milk mixture 319 may be dried to generate a dried base powder 324. In some embodiments, a dried base powder 324 may be reconstituted (e.g., by adding water) to generate one or more of an electrolyte drink 322, a reconstituted milk mixture 319 and a reconstituted milk base 312.

A drying procedure 126, in some embodiments, may reduce the moisture content of a milk base 312 and/or a milk mixture 319 to a desired level (e.g., lower moisture content, a desired moisture content). A moisture content of a dried base powder 324 may be, for example, below about 95%, or below about 90%, or below about 80%, or below about 70%, or below about 60%, or below about 50%, or below about 40%, or below about 30%, or below about 20%, or below about 10%, or below about 5%, or below about 1% by weight of the dried base powder 324, in some embodiments. A drying procedure 126 may be performed using a mechanism including, for example, a spray dryer (preferred), a drum dryer, a double drum dryer, flash dryer, a fluid-bed dryer, a convection dryer, an evaporator, or any combination thereof.

In some embodiments, an inlet temperature of a dryer mechanism (the temperature at the entrance to a dryer) may be above 25° C., or above 50° C., or above 75° C., or above 100° C., or above 125° C., or above 150° C., or above 175° C., or above 200° C., or above 225° C., or above 250° C., or above 275° C., or above 300° C., or above 325° C., or above 350° C., or above 375° C., or above 400° C., or above 425° C., or above 450° C., or above 475° C., or above 500° C. An inlet temperature, in some embodiments, may be from about 25° C. to about 50° C., or from about 50° C. to about 75° C., or from about 75° C. to about 100° C., or from about 100° C. to about 125° C., or from about 125° C. to about 150° C., or from about 150° C. to about 175° C., or from about 175° C. to about 200° C., or from about 200° C. to about 225° C., or from about 225° C. to about 250° C., or from about 250° C. to about 275° C., or from about 275° C. to about 300° C., or from about 300° C. to about 325° C., or from about 325° C. to about 350° C., or from about 350° C. to about 375° C., or from about 375° C. to about 400° C., or from about 400° C. to about 425° C., or from about 425° C. to about 450° C., or from about 450° C. to about 475° C., or from about 475° C. to about 500° C., or above 500° C. An inlet temperature may be from about 50° C. to about 100° C., or from about 100° C. to about 150° C., or from about 150° C. to about 200° C., or from about 200° C. to about 250° C., or from about 250° C. to about 300° C., or from about 300° C. to about 350° C., or from about 350° C. to about 400° C., or from about 400° C. to about 450° C., or from about 450° C. to about 500° C., or above 500° C., in some embodiments. According to some embodiments, an inlet temperature of a dryer mechanism may be about 225° C.

According to some embodiments, an outlet temperature of a dryer mechanism (the temperature at the exit from a dryer) may be below about 300° C., or below about 275° C., or below about 250° C., or below about 225° C., or below about 200° C., or below about 175° C., or below about 150° C., or below about 125° C., or below about 100° C., or below about 75° C., or below about 50° C., or below about 25° C. An outlet temperature may be from about 300°

C. to about 275° C., or from about 275° C. to about 250° C., or from about 250° C. to about 225° C., or from about 225° C. to about 200° C., or from about 200° C. to about 175° C., or from about 175° C. to about 150° C., or from about 150° C. to about 125° C., or from about 125° C. to about 100 ° C., or from about 100° C. to about 75° C., or from about 75° C. to about 50° C., or from about 50° C. to about 25° C., or below about 25° C., in some embodiments. An outlet temperature, in some embodiments, may be from about 300° C. to about 250° C., or from about 250° C. to about 200° C., or from about 200° C. to about 150° C., or from about 150° C. to about 100° C., from about 100° C. to about 50° C., or from about 50° C. to about 25° C., or below about 25° C. According to some embodiments, an outlet temperature of a dryer mechanism may be about 75° C.

### Formulating a Milk Base to Generate a Microcrop Milk

A method of producing a microcrop milk 313 may comprise formulating 123 a milk base 312 to produce a microcrop milk 313. Formulating 123 a milk base 312 to produce a microcrop milk 313 may include one or more of amending a milk base 312 and homogenization of a milk base (e.g., an amended milk base).

According to some embodiments, amending a milk base 312 to produce an amended milk base may include adding one or more formulation additives 210. A formulation additive 210 may include water, one or more fat components, one or more emulsifiers, one or more enhancers, or any combination thereof.

According to some embodiments, amending a milk base 312 to produce an amended milk base may include adding a fat component to produce a microcrop milk 313 with a consistency that is closer to a more traditional dairy-based milk product. A fat component may comprise one or more of palm oil, coconut oil, avocado oil, almond oil, hemp oil, soybean oil, sunflower oil, canola oil, oat oil, cashew oil, peanut oil, pea oil, quinoa oil, rice bran oil, barely oil, or other plant- or nut-based oil. In some embodiments, a fat component may comprise a dairy-based fat such as milk fat from a mammalian milk According to some embodiments, a fat component may include omega-3 oils derived from marine organisms such as fish, krill, and algae. In some embodiments, a fat component may have animal origin (e.g., butterfat) or be a fat substitute (e.g., low or zero calorie). Persons having skill in the art would understand that the any number of animal based, plant based, and artificial fats may be used to amend a milk base 312 without deviating from the present disclosure. Moreover, various forms of fat additives (e.g., bulk and powdered fats) are within the scope of the present disclosure.

Amending a milk base 312 to produce an amended milk base may include adding one or more emulgents (i.e., emulsifier, emulsifying agent) to a milk base to produce a microcrop milk 313 that is less likely to separate into a fat component and milk base after an addition of a fat component and homogenization. An emulsifier may comprise, without limitation, one or more of mustard, soy lecithin, egg lecithin, monoglycerides, diglycerides, polysorbates, carrageenan, guar gum, canola oil, calcium stearoyl-2-lactylate. polyglycerol esters, sorbitan esters, PG esters, sugar esters, acetylated monoglycerides, lactylated monoglycerides, or other plant-derived, animal-derived, or synthetic emulsifiers.

In some embodiments, amending a milk base 312 may include adding one or more enhancers. Enhancers (e.g., flavoring, color augmentator) may be added to the milk base 312 to alter (e.g., improve) the flavor, taste, mouth feel, smell, appearance, or useful life of the microcrop milk 313 Enhancers may comprise any combination of flavors, dyes, or other agents, which are meant to improve the flavor, taste, mouth feel, smell, appearance, or shelf-life of the microcrop milk 313. By way of example, and not limitation, enhancers may include sugars, natural and artificial coloring agents, honey, monosodium glutamate, salts, nut oils, diary or non-dairy milks, artificial sweetening substances, color retention agents, thickeners, preservatives, and other natural or artificial flavoring or coloring agents. Addition of enhancers may occur before or after homogenization. In some embodiments, enhancers are not added at all.

Formulating 123 a milk base 312 to produce a microcrop milk 313 may include homogenization of a milk base (e.g., an amended milk base). Homogenization may be achieved by using one or more of: homogenizers, homogenization valves, extruders, hammer mills, colloid mills, or other means of converting two immiscible liquids (e.g., a milk base 312 and fat component) into an emulsion.

Formulating 123 a milk base 312 may additionally include a treatment (e.g., treatment 110) to decrease a microbial population or enzymatic load of a milk base 312. As described above, a treatment may be performed using any method (e.g., pasteurization, heating to specific temperatures and maintaining said temperature for a specific time, gamma ray exposure) suitable for decreasing a microbial population or enzymatic load of a milk base 312 to a desired level without resulting in unacceptable levels of degradation to a soluble protein present in a milk base 312.

Persons skilled in the art may make various changes without departing from the scope of the instant disclosure. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. Where desired, some embodiments of the disclosure may be practiced to the exclusion of other embodiments.

Also, where ranges have been provided, the disclosed endpoints may be treated as exact and/or approximations (e.g., read without or with “about”) as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In some embodiments, variation may simply be +/−10% of the specified value. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/−about 10%, depicted value +/−about 50%, depicted value +/−about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

What is claimed is:

1. A method of treating a first juice derived from a microcrop to generate a product comprising microcrop protein, the method comprising:
hydrolyzing the first juice, using one or more enzymes, to generate a hydrolyzed protein juice; and
filtering the hydrolyzed protein juice to generate at least one of a decolored permeate and a green retentate.

2. The method of claim 1, further comprising:
demineralizing the decolored permeate to generate a demineralized stream;
dewatering the demineralized stream to generate a dewatered demineralized stream; and
generating a milk mixture, wherein the milk mixture comprises at least a portion of the dewatered demineralized stream.

3. The method of claim 2, further comprising reducing a concentration of polyphenols in the decolored permeate using adsorption.

4. The method of claim 2, further comprising:
mixing at least a portion of the dewatered demineralized stream with at least a portion of the demineralized stream to generate the milk mixture.

5. The method of claim 2, further comprising dewatering the milk mixture to generate a milk base.

6. The method of claim 5, further comprising formulating the milk base to generate a microcrop milk.

7. The method of claim 6, wherein formulating the milk base comprises amending the milk base with a fat component and an emulsifier.

8. The method of claim 2, further comprising drying the milk mixture to generate a dried base powder.

9. The method of claim 4, further comprising drying the milk mixture to generate a dried base powder.

10. The method of claim 5, further comprising drying the milk base to generate a dried base powder.

11. The method of claim 1, further comprising:
treating the first juice, wherein treating comprises at least one of pasteurization, heating to specific temperatures and maintaining said temperature for a specific time, gamma ray exposure; and
cooling the treated first juice to an incubation temperature.

12. The method of claim 1, further comprising:
dewatering the green retentate to generate a dewatered green retentate; and
drying the dewatered green retentate to generate a green food product.

13. The method of claim 12, further comprising treating the dewatered green retentate, wherein treating comprises at least one of pasteurization, heating to specific temperatures and maintaining said temperature for a specific time, and gamma ray exposure.

14. The method of claim 1, further comprising:
demineralizing the decolored permeate to generate a demineralized stream;
dewatering the demineralized stream to generate a dewatered demineralized stream; and
drying the dewatered demineralized stream to generate a colorless protein product.

15. The method of claim 14, further comprising reducing a concentration of polyphenols in the decolored permeate using adsorption.

16. The method of claim 14, further comprising treating the dewatered demineralized stream, wherein treating comprises at least one of pasteurization, heating to specific temperatures and maintaining said temperature for a specific time, and gamma ray exposure.

17. The method of claim 1, further comprising heating the first juice to an incubation temperature.

18. The method of claim 1, further comprising generating the first juice.

19. The method of claim 18, wherein generating the first juice comprises:
lysing a microcrop to generate a lysed biomass;
separating the lysed biomass to generate a solid fraction and a juice fraction; and
separating the juice fraction to generate the first juice.

20. The method of claim 19, further comprising hydrolyzing the lysed biomass prior to separating the lysed biomass to generate a solid fraction and a juice fraction.
